(19) United States
(12) Patent Application Publication
(10) Pub. No.: US 2024/0299535 A1
(43) Pub. Date: Sep. 12, 2024

(54) TARGETED GLYCOSAMINOGLYCAN-PARTICLES AND METHODS OF USE

(71) Applicant: The Board of Regents of the University of Oklahoma, Norman, OK (US)

(72) Inventors: Paul L. DeAngelis, Norman, OK (US); Dixy E. Green, Norman, OK (US); Stefan Wilhelm, Norman, OK (US); Wen Yang, Norman, OK (US)

(21) Appl. No.: 18/579,096

(22) PCT Filed: Jul. 13, 2022
(86) PCT No.: PCT/US2022/037007

§ 371 Date: Jan. 12, 2024

Related U.S. Application Data
(60) Provisional application No. 63/222,622, filed on Jul. 16, 2021.

(51) Int. Cl. A61K 39/385; A61K 39/00; A61K 47/61; A61K 47/69

(52) U.S. Cl. CPC A61K 39/385; A61K 47/61; A61K 47/6929; A61K 2039/53; A61K 2039/6087

(57) ABSTRACT

Compositions containing compositions of heparosan polymers linked to a particle, such as a metallic or polymeric or lipid-containing nanoparticle are described, for use in cell delivery applications.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, such as U.S National Phase Application under 35 U.S.C. § 371 of International Application No. PCT/US2022/037007, filed on Jul. 13, 2022 and U.S. Provisional Application No. 63/222,622, filed Jul. 16, 2021 are hereby incorporated by reference in their entirety.

### BACKGROUND

Certain polymers, including poly[ethylene glycol] (PEG) have been approved by the Food and Drug Administration (FDA) for use as part of therapeutic compositions. In addition, we have described Heparosan (HEP; N-acetylheparosan) as an agent that can provide some therapeutic benefits when linked to different types of drug compositions (U.S. Pat. No. 9,925,209 issued on Mar. 27, 2018).

Nanoparticles provide flexible platforms for the development of drug delivery technologies, disease diagnostics, and vaccines. Yet, upon exposure to physiological fluids, proteins adsorb onto the nanoparticle surface to form a layer termed the protein corona. This protein corona can alter the biological fate and immunogenicity of nanoparticles. For example, certain proteins can undergo configurational changes upon adsorption to nanoparticle surfaces, potentially resulting in nanoparticle aggregation or the presentation of novel antigenic sites. To address this challenge, nanoparticle surface modifications with synthetic polymers have been used in nanomedicine to enhance colloidal stability and reduce the non-specific protein adsorption.

While the FDA has approved the clinical use of nanoparticles with polymer coatings, such as poly(ethylene glycol) (PEG) and dextran, these coating agents have been reported in some cases to adversely impact nanomedicine safety and efficacy. These reports have raised growing clinical concern about anti-PEG immunogenicity, which may be amplified by the widespread use of PEG in cosmetics, health care products, and over-the-counter medications as well as the recently introduced COVID-19 vaccines employing PEG-based nanoparticles. Anti-PEG antibodies can bind to PEGylated nanoparticles, which may induce undesired immune responses, including premature clearance of nanomedicines, allergic reactions, and anaphylaxis.

### BRIEF DESCRIPTION OF THE DRAWINGS

FIG. 1 is a schematic diagram of a model of heparosan-coated nanoparticle being incubated with an antigen presenting cell and showing low adsorption to serum proteins.

FIG. 2 is a schematic of the process of methods for attaching heparosan to a gold nanoparticle (AuNP). Process (A) shows the general surface attachment strategy of OPSS-terminated HEP (OPSS-HEP). Process (B) shows Salt aging method wherein (i) OPSS-HEP is mixed with colloidally dispersed citrate-coated AuNPs and (ii) the ionic strength of the dispersion is then increased by the step-wise addition of a NaCl solution (denoted with multiple arrows). Process (C) is a pH method wherein (i) OPSS-HEP is mixed with colloidally dispersed citrate-coated AuNPs and (ii) the pH of the colloidal dispersion is subsequently decreased to approximately pH 3 by the one-step addition of a hydrochloric acid solution.

FIG. 3 is a set of panels showing characterization of heparosan (HEP) surface modification using 15-nm AuNPs. Panel (A) shows that dynamic light scattering (DLS) was used to measure the hydrodynamic diameter of 15-nm AuNPs after mixing with various amounts of HEP per nm² of nanoparticle surface area with vortexing (without changes in salt concentration (salt aging) or pH reduction). The increase in hydrodynamic diameter of only ˜3 nm suggested that HEP did not efficiently conjugate AuNPs. Bars indicate mean±SD (n=3). Panel (B) shows the DLS results of 15-nm AuNPs mixed with various amounts of HEP per nm² nanoparticle surface area and addition of saline (salt aging) or subsequent decrease in pH (pH reduction method). The increase in hydrodynamic diameter of ˜49 nm suggested efficient HEP conjugation. Bars indicate mean±SD (n=3). Statistical tests were performed by two-way ANOVA; n.s. indicates no statistically significant differences. Panels (C-D) shown representative TEM micrographs of 15-nm citrate-coated AuNPs with a diameter of 14.9±0.6 nm (Panel C) and HEP-AuNPs with a diameter of 24.7±1.4 nm (Panel D). Citrate-coated AuNPs (Panel C) and HEP-AuNPs (Panel D) were stained with 2% uranyl acetate. The light grey halo around the dark AuNP core corresponds to the HEP coating or shell of the surface in Panel D. Scale bar indicates 50 nm. Panel (E) shows a size analysis of 15 nm citrate- and HEP-AuNPs by TEM imaging. The core only is the core size of citrate-AuNPs of Panel C (control; red bar). The core of HEP-AuNPs of Panel D is represented by a brown bar. The diameter of the core and shell of HEP-AuNPs of panel D is represented by a slanted lined brown bar. Bars indicate mean±SD. Statistical tests were performed by one-way ANOVA (p<0.0001 (****); n.s. indicates no statistically significant differences). Panel (F) is a schematic of AuNP surface modification with radiolabeled HEP. Panel (G) shows a line graph of a radiochemical assessment of HEP coating density. Liquid scintillation analysis was used to measure the ³H radioactivity in comparison to coating density (the addition of [³H]-HEP per nm²) conjugated to 15-nm AuNPs.

FIG. 4 shows nanoparticle surface engineering with heparosan reduces protein corona formation. Panel (A) is a schematic representation of nanoparticle protein corona formation with and without HEP coating. Panels (B) and (C) show that dynamic light scattering (DLS) was used to compare the hydrodynamic diameter differences before and after FBS incubation (slanted lined bars stand for incubation with FBS) of citrate-coated (B; 0 HEP/nm²) and HEP-coated (Panel C) 15-, 55-, or 100-nm AuNPs. Bar graphs indicate mean±SD (n=3). Statistical tests were performed by two-way ANOVA (p<0.0001). Panel (D) shows the quantitative BCA protein assay results for HEP- or PEG-coated 55-nm AuNPs created by increasing amounts of polymer added to coating reactions. Results are presented as mean±SD (n=3). Panels (E) and (F) are SDS-PAGE gels showing the qualitative biomolecular composition of the adsorbed FBS protein layer on 55-nm AuNPs with various surface HEP (Panel E) or PEG densities (Panel F). The coating densities represent the added amount of polymers in a coating reaction per nanoparticle surface area.

FIG. 5 shows the cytotoxicity, hemolysis, and cell uptake of HEP- and PEG-modified 55-nm AuNPs. Panel (A) shows a cell viability test of RAW 264. 7 or J774A macrophages treated with 1 nanoMolar HEP-AuNPs for 48 h with control groups (cells with PEG-AuNPs or without AuNPs) by XTT assay. Bar graphs indicate mean±SD (n=5). Panel (B) shows the results of a hemolysis assay of 55-nm nanoparticles (1-nM HEP- or PEG-AuNPs final). 1×PBS or 1% Triton-X 100 were used as negative and positive controls, respectively. Bar graphs indicate mean±SD (n=3). Panel (C) shows the results of cell uptake assays: HEP-AuNPs or control PEG-AuNPs were incubated with B16F10 murine melanoma, C2C12 murine muscle cells, J774A.1 murine macrophages, RAW 264.7 murine macrophages, or DC2.4 murine dendritic cells. ICP-MS was performed to quantify cell uptake of nanoparticles. About 70×, 230×, and 45× more HEP-AuNPs were internalized than PEG-AuNPs in J774A.1 macrophage, RAW 264.7 macrophage, and DC 2.4 dendric cells, respectively. Bar graphs indicate mean±SD (n=3-4). Panel (D) shows the effect of FBS (protein corona) on cellular uptake of HEP-AuNPs with control (PEG-AuNPs) when incubated with J774A.1 murine macrophages, RAW 264.7 murine macrophages, and DC2.4 murine dendritic cells. ICP-MS was performed to quantify nanoparticles cell uptake. No significant difference was observed with FBS-treatment as in panel C. Bar graphs indicate mean±SD (n=3-4). Panel (E) shows confocal laser scanning microscopy images of HEP- and PEG-coated AuNPs incubated with DC2.4 dendritic cells for 3 hours. The added coating reagents for the preparations of HEP-AuNPs and PEG-AuNPs in this figure was ˜5 polymers/nm². The scale bar indicates 20 μm.

FIG. 6 shows that the cellular uptake of heparosan (HEP) modified gold nanoparticles (AuNPs) is time-dependent. Panel (A) is representative brightfield light micrographs of HEP-AuNPs internalization in RAW 264.7 macrophages at 0 h, 1 h, 3 h, and 9 h. Scale bar: 50 m. Panel (B) is ICP-MS results of 55-nm HEP-AuNPs uptake in RAW 264.7 macrophages over time. The data points indicate mean values and standard deviation (n=3-4). Panel (C) is real-time confocal laser scanning microscopy (CLSM) imaging of HEP-AuNP internalization in live RAW 264.7 macrophages. Scale bars: 20 m. Panel (D) shows a representative individual cell image which was selected from Panel (C). The right panel shows the AuNPs channel. Scale bars: 10 m. Panel (E) is transmission electron micrographs of 55-nm HEP-AuNP internalization in RAW 264.7 after 3 h, 6 h, and 24 h incubation. The insert at the bottom right corner of each micrograph shows a higher magnification view of the selected field of view sections. Scale bars: 500 nm.

FIG. 7 shows that HEP-coated nanoparticles enter innate immune cells through an endocytotic pathway. Panel (A) is a schematic representation of the uptake pathway study: (i) non-specific endocytosis inhibition to determine whether nanoparticle cellular uptake is energy-dependent. (ii-iv) Specific endocytosis inhibitors for studying (ii) caveolae-mediated endocytosis, (iii) clathrin-mediated endocytosis, and (iv) macropinocytosis. Panels (B) to (D) are ICP-MS quantification of the nanoparticle cellular uptake in RAW 264.7 macrophages at 4° C.—Panel (B), in the presence of ATPase inhibitor sodium azide—Panel (C), or chemical endocytosis inhibitors of caveolae-mediated endocytosis, clathrin-mediated endocytosis, and micropinocytosis—Panel (D). AuNPs modified with 13-kDa HEP (at 0.2 nM) were used as control without inhibitors at 37° C. Bars indicate mean±SD (n=3-4); statistical tests used one-way ANOVA (p<0.0001 (****); p<0.0021 (**); p<0.0332 (*).

FIG. 8 shows nanoparticle surface coating with HEP promotes multivalent interactions with innate immune cells. Panel (A) shows a schematic representation of the surface coating process wherein (i) the HEP polymers were added to the AuNPs with theoretical surface coating densities ranging from 0 to 14 HEP/nm² and (ii) backfilling of the nanoparticle surface was achieved by adding a constant saturating amount of PEG (adding the equivalent of 7 PEG/nm²) to generate HEP/PEG-AuNPs. Panel (B) shows that the uptake efficiency was measured as a function of surface HEP density by ICP-MS. The data points indicate mean values±SD (n=3-4). Panels (C) to (E) are representative brightfield light micrographs of HEP/PEG-AuNPs in cells with the dark spots within cells indicating nanoparticle accumulation. The inserted bar graphs within Panels (C) to (E) display the quantitative ICP-MS results of nanoparticle cell uptake. The data points indicate mean values±SD (n=3-4). Scale bar: 50 μm.

FIG. 9 is a bar chart demonstrating that nanoparticles coated with a variety of GAGs, including hyaluronan (HA) and some versions of chondroitin sulfate (CS), can be internalized by immune cells according to embodiments. On the other hand, PEG-, mannan- and dextran-coated NPs were much less efficiently endocytosed.

### SUMMARY

One embodiment relates to a composition having at least one GAG-particle. The composition may include at least one GAG polymer; a particle capable of carrying an immunogenic or immune-response modulating molecule; and at least one immunogenic molecule or immune-response modulating molecule covalently or non-covalently linked to the particle.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

One embodiment is a composition having at least one glycosaminoglycan polymer bound to a particle; and at least one immunogenic molecule covalently or non-covalently linked to the particle.

It is to be understood that any features of the systems disclosed herein may be combined together in any desirable manner and/or configuration. Further, it is to be understood that any features of the methods disclosed herein may be combined together in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

### DETAILED DESCRIPTION

Before explaining at least one embodiment of the presently disclosed and/or claimed concept(s) in detail, it is to be understood that the presently disclosed and/or claimed concept(s) is not limited in its application to the details of construction and the arrangement of the components or steps or methodologies set forth in the following description or illustrated in the drawings. The presently disclosed and/or claimed concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and/or claimed concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those of ordinary skill in the art to which this presently disclosed and/or claimed concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

Polysaccharides are large carbohydrate molecules comprising from about 20 sugar units to thousands of sugar units. Oligosaccharides are smaller carbohydrate molecules comprising less than about 20 sugar units. Animals, plants, fungi and bacteria produce an enormous variety of polysaccharide structures that are involved in numerous important biological functions such as structural elements, energy storage, and cellular interaction mediation. Often, the polysaccharide's biological function is due to the interaction of the polysaccharide with proteins such as receptors and growth factors. The glycosaminoglycan (GAG) family includes negatively charged polysaccharides and oligosaccharides which may be used in the body for cell signaling. The four primary groups of GAGs are classified based on their core disaccharide units and include heparin/heparan sulfate, chondroitin sulfate/dermatan sulfate, keratan sulfate, and hyaluronic acid. Another GAG is heparosan, a bioprecursor in the natural biosynthesis of heparin and heparan sulfate.

Embodiments relate to the use of nanoparticles coated with a GAG molecule to be used to deliver immunogenic molecules, including polypeptides, proteins, peptides, or nucleic acids encoding such immunogenic molecules to immune cells. The GAG molecule may be chemically or physically linked to the nanoparticles, which in turn may contain the immunogenic molecule to be delivered to an immune cell and released into the cytoplasm and/or other intracellular compartments to create an immunogenic response.

For example, embodiments may relate to a GAG molecule, such as heparosan, linked to a particle, such as a liposome, which contains a peptide that is known to be displayed on the surface of an immune system cell, such as a macrophage or dendritic cell. The GAG molecule/liposome/immunogenic peptide complex may be delivered in vivo or ex vivo to a mammal to generate an immune response to the immunogenic molecule. The complex may therefore act as a vaccine to generate an immune response in the mammal against the immunogenic peptide.

In another embodiment, the GAG molecule may be linked to a particle and used as a contrast agent to visualize immune cells within the body. For example, a heparosan polymer may be bound to the surface of a gold or silver particle and used as a contrast agent. As described below, heparosan/metal particle complexes were found to be readily taken up by immune system cells, such as macrophages and dendritic cells. Thus, one could visualize areas of inflammation or other centers of immune cells by giving a patient a heparosan/metal particle complex, such as heparosan/Au which may be seen on certain types of imaging systems.

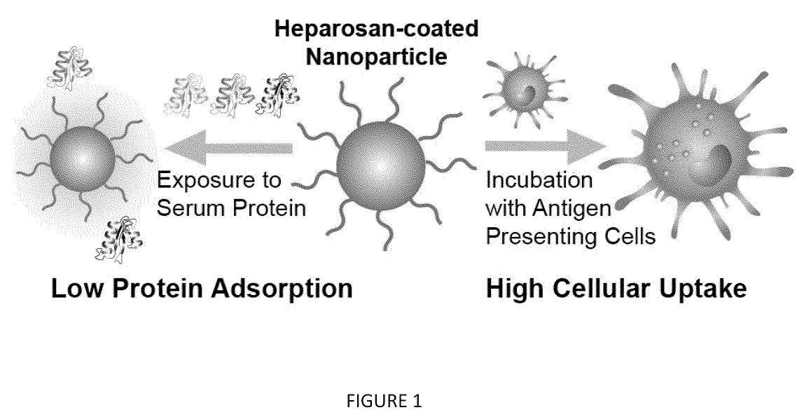

As shown in FIG. 1, embodiments relate to the preparation and use of certain sugar-modified particles (e.g., GAG-modified nanoparticles, colloids, molecular suspensions, etc. with a payload, e.g., immunogenic molecule, mRNA, DNA, etc.) that can selectively target immune cell types, be internalized, and then traffic inside the cell's compartments (e.g., endosomes, lysosomes, endoplasmic reticulum, nucleus, etc) or escape into the cytoplasm to elicit an intended biological/therapeutic effect. In one example, particles linked to heparosan (a glycosaminoglycan that is the unmodified precursor of heparin; [-4-GlcA-1beta-4-GlcNAc-lalpha-]ₙ) were found to be selectively taken up by some cell types of the immune system, but not other cell types, and then transit from intracellular vesicles to the cytoplasm. As shown in FIG. 1, a heparosan coated nanoparticle can be incubated with antigen presenting cells and have high cellular uptake. The coated nanoparticle may also have an immunogenic molecule attached to the nanoparticle which is then taken into the antigen presenting cell with high efficiency. In addition, the heparosan coated nanoparticle has low adsorption to other serum proteins, which would lead to less nanoparticle clearance or being taken up by non-targeted cell types.

Compositions of a GAG molecule linked to such modified particles may be useful for vaccines (e.g., mRNA-based or protein-based versions to treat COVID-19 and other diseases) that need to be delivered to the cytoplasm or inner compartments of particular cells for action in the body. For example, in one embodiment, the particle may be a nanoparticle that includes a messenger RNA molecule which encodes the spike protein of the SARS-COV-2 virus or its mutants. In another embodiment, the particle may be a nanoparticle that includes the spike protein or a fragment of the SARS-COV-2 virus or its mutants.

We discovered that such GAG-coated particles were found to have a much higher particle uptake than current standard polymer-modified particles, such as particles modified with polyethylene glycol (PEG) or the carbohydrates mannan or dextran. Our other discovery was that HEP-particle compositions had selectivity for certain target cell types, such as dendritic cells, macrophages, and certain white blood cells but were not taken up well by other cell types. This makes such HEP-particle compositions useful for creating vaccines by being delivered to such target cell types and stimulating an immune response in a patient.

### Making Heparosan

Heparosan ([-4-N-acetylglucosamine-α1,4-glucuronic acid-β1-]ₙ) is a natural GAG polysaccharide structurally related to heparin. The heparosan chain is very hydrophilic due its two hydroxyl groups on every monosaccharide unit and a negative carboxylate group on every other monosaccharide unit. The heparosan molecule is neither decorated with sulfate groups nor epimerized at glucuronic acid residues; thus, heparosan is relatively biologically inactive to a significant extent with respect to coagulation (i.e. clotting factors not activated to a significant extent), modulation of proliferation (i.e. growth factors do not bind to a significant extent), inflammation (i.e. cytokines do not interact to a significant extent), and a plethora of other activities (Capila et al. (2002) Angew Chem Int Ed Engl, 41(3):391-412).

In one embodiment, making heparosan may utilize a synchronized, stoichiometrically-controlled reaction employing a sugar-polymerizing enzyme (e.g., PmHS1 or PmHS2 and combinations thereof or similar analogs with roughly equivalent biological activity) in an aqueous buffer system that results in a quasi-monodisperse (very narrow size distribution) product. The heparosan synthase can be utilized in vitro to synthesize quasi-monodisperse (i.e. very narrow size distributions approaching the ideal polydispersity value of ‘1’) polymer preparations (Sismey-Ragatz et al. (2007) J. Biol. Chem., 282(39):28321-28327) with homogenous reactive end-groups for coupling to biologic targets or delivery vehicles. The narrow size distribution is achieved by synchronizing the polymerization reaction using a primer, typically a short heparosan fragment, which allows the normal slow chain initiation step of biosynthesis to be bypassed. Therefore, all polymers are rapidly extended by PmHS1 (or similar enzyme constructs and mutants) in a virtually parallel fashion; thus, all final chains have a very similar length. No post-polymerization purification for size control is required. The primer also allows the contribution of the unique reactive group(s), if desired, that helps assure that every polymer chain can be activated for immunogenic molecule or vehicle coupling. In addition, the primer position in the heparosan chain at the reducing terminus does not interfere with lysosomal degradation allowing the heparosan chain to be digested to a tiny stub containing the linker site used for immunogenic molecule attachment; this stub is usually excreted in the urine or feces.

The chain size or molecular weight of any particular heparosan preparation is controlled by manipulating the stoichiometric ratio of the primer to the UDP-sugar precursor. Basically, for a given amount of UDP-sugars, a low concentration of primer yields longer chains while, on the other hand, a high primer concentration yields shorter chains (of course, the former case has fewer moles of product formed than the latter). Heparosan molecules in the range of from about 10 kDa to about 4,500 kDa (or from about 50 to about 22,500 monosaccharide units) have been synthesized by the synchronized chemoenzymatic method, thus potentially accessing a wider useful size range than possible for PSA, HES, or PEG. By using other methods such as step-wise synthesis (elongation with a pair of PmHSl mutants or catalytically similar enzymes) or chemical fragmentation (e.g., acid or oxidative cleavage) or enzymatic fragmentation (e.g., heparanase, heparin lyases), heparosan polymers from about 600 Da to about 10 kDa can be made.

In addition to in vitro chemoenzymatically produced heparosan, the same [-4-N-acetylglucosamine-α1,4-glucuronic acid-β1-]ₙ polymeric structure may also be produced in vivo by the culture or fermentation of certain microbes, but the polymer may not be as monodisperse or as easily activated for coupling in comparison to the in vitro produced polymer. Some examples of the fermentation systems include natural heparosan producers including Pasteurella multocida or allies (e.g., Avibacterium species), Escherichia coli K5, or the recombinant versions (e.g., Gram − or Gram + bacteria, Achaea, or eukaryotic hosts) expressing the heparosan biosynthetic machinery (e.g., synthases or polymerases or glycosyltransferases, UDP-glucose dehydrogenases, etc.) of the natural heparosan-producing species. The microbial heparosan production route may be used to make polymer for preparation of HEP-particle compositions, and is covered by the spirit and scope of the presently disclosed and/or claimed inventive concept(s); the source of heparosan polymer is not important for the enhancement of therapeutic efficacy.

Certain embodiments of the presently disclosed and/or claimed inventive concept(s) are directed to a method for preparing a pharmaceutically active composition comprising a GAG, such as heparosan, linked to a nanoparticle, such as a metal (e.g., gold or silver) or polymeric or a lipid-based nanoparticle or other inorganic and/or organic materials.

The GAG polymer used in the method of making the GAG-particle compositions may be characterized as being substantially non-antigenic, substantially non-immunogenic, and substantially biologically inert within extracellular compartments of a mammalian patient, being stable in the mammalian bloodstream, and/or being degraded intracellularly in the mammalian patient. The GAG polymer may be produced by any method known in the art or otherwise contemplated herein, as will be discussed in greater detail herein below. In addition, one of the advantages of the presently disclosed and/or claimed inventive concept(s) is that the GAG polymer can be synthesized in a reproducible, and defined manner so as to provide all of the advantages of PEG without its potential side effects.

In certain particular, non-limiting embodiments, the GAG polymer may have a mass in a range of from about 600 Da to about 4.5 MDa. In addition, in certain particular, non-limiting embodiments, the GAG polymers may be polydisperse in size. Alternatively, in other non-limiting embodiments, the substantially monodisperse in size. For example, but not by way of limitation, the substantially monodisperse GAG polymers may be heparosan polymers and may have: (a) a molecular weight in a range of from about 1 kDa to about 0.5 MDa and a polydispersity value in a range of from about 1.0 to about 1.1; (b) a molecular weight in a range of from about 0.5 MDa to about 4.5 MDa and a polydispersity value in a range of from about 1.0 to about 1.5; and (c) a molecular weight in a range of from about 0.5 MDa to about 4.5 MDa and a polydispersity value in a range of from about 1.0 to about 1.2.

In certain embodiments of the presently disclosed and/or claimed inventive concept(s), the GAG polymer utilized in the methods may be a linear chain. Alternatively, the heparosan polymer may have a branched geometry. In addition, the GAG polymer may have a dendritic geometry. If the GAG polymer is heparosan, it may be unsulfated and unepimerized. For hyaluronan (hyaluronic acid, HA) and unsulfated chondroitin, similar technology to the above mentioned heparosan for producing the GAG is available in vitro or in vivo. For chondroitin sulfates (CS), typically various extracts of tissues (e.g., mammalian trachea, shark fin, squid cartilage) are used to prepare the GAG. In addition, starting with unsulfated chondroitin and then treating with chemical reagents (e.g., sulfur trioxide complexes, chlorosulfonic acid) and/or sulfotransferase enzymes are other routes to prepare CS. Likewise, for heparan sulfate or heparin, the same general routes as CS are possible. For our invention, the source of GAG is not critical as long as the targeting and internalization effect is retained.

Furthermore, this invention describes GAG-coated modified particles with multiple sugar polymers per particle as the internalization of these constructs was found to be very efficient in certain cells. In particular, high density heparosan-coated particles were found to have increased internalization into immune cells in comparison with particles where a single or a few HEP polymers are attached to a macromolecule. In the latter case, it appears that cells do not greatly internalize the monovalent, or sparsely decorated, HEP-macromolecule conjugates; instead the sugar chain acts as a stealth agent, not a targeted delivery agent as indicated by the long half-life of such conjugates or free heparosan chains in plasma upon administration into animals (Jing W. Roberts J W, Green D E, Almond A, DeAngeli P L. Synthesis and characterization of heparosan-granulocyte-colony stimulating factor conjugates: a natural sugar-based drug delivery system to treat neutropenia. Glycobiology. 2017; 27(11):1052-1061).

The linkage between the nanoparticle and the at least one GAG polymer may be substantially stable or substantially labile. The use of a substantially labile bond (e.g., ester, disulfide) allows for release of the GAG polymer from the nanoparticle within the cytoplasm or an inner compartment of a cell. In certain embodiments, the immunogenic molecule may be integrated into the nanoparticle, such as a peptide or nucleic acid molecule within a liponanoparticle or a polymeric particle (e.g., PLGA, PLA). In some embodiments, the immunogenic molecule may be enclosed within the lumen of a liposome (e.g., water-soluble molecules) and/or within its lipid bilayer (e.g., hydrophobic molecules). In one embodiment the immunogenic molecule may be at least one polypeptide, such as but not limited to, a peptide, a protein, and/or a glycoprotein. In other non-limiting embodiments, the immune modulating system may be at least one polymer comprised of deoxyribonucleic acid and/or ribonucleic acid encoding an immunogenic or a regulatory molecule. In one embodiment, the composition may be a vaccine, such as a nucleic acid vaccine which encodes an antigen after entering the cell and being released into the cytoplasm. In one embodiment, the composition is an RNA vaccine encoding a pathogen molecule designed to provide immunity to a virus, such as the SARS-CoV-2 virus or its mutants responsible for COVID-19 disease.

The nanoparticle-GAG polymer composition may be administered, for example but not by way of limitation, parenterally, intraperitoneally, intraspinally, intravenously, intramuscularly, intrathecally, intravaginally, subcutaneously, intranasally, rectally, and/or intracerebrally. Dispersions of the GAG-modified nanoparticles may be prepared in saline, glycerol, liquid poly[ethylene glycols], and mixtures thereof, as well as in oils. Under ordinary conditions of storage and use, such preparations of the composition may also contain a preservative to prevent the growth of microorganisms.

Pharmaceutical compositions suitable for injection use include sterile aqueous solutions (where water soluble) or dispersions and sterile powders for the extemporaneous preparation of sterile injectable solutions or dispersions. When used for injection, the composition should be sterile and should be fluid to the extent that easy syringability exists. The compositions should also be stable under the conditions of manufacture and storage and should be preserved against the contaminating action of microorganisms such as bacteria and fungi. The immunogenic molecule-GAG polymer particles may be used in conjunction with a solvent or dispersion medium containing, for example but not by way of limitation, water, ethanol, poly-cl (i.e. glycerol, propylene glycol, and liquid poly[ethylene glycol], and the like), suitable mixtures thereof, vegetable oils, and combinations thereof.

Sterile injectable solutions may be prepared by incorporating the composition in the required amount in an appropriate solvent with one or a combination of ingredients enumerated above, as required, followed by filtered sterilization. Generally, dispersions are prepared by incorporating the heparosan-modified nanoparticles into a sterile carrier that contains a basic dispersion medium and the required other ingredients from those enumerated above. In the case of sterile powders for the preparation of sterile injectable solutions, the methods of preparation may include vacuum drying, spray drying, spray freezing, and/or freeze-drying that yields a powder of the active ingredient (i.e., the HEP-particle composition) plus any additional desired ingredient from a previously sterile-filtered solution thereof.

In certain embodiments it may be desired to formulate parenteral compositions in dosage unit form for ease of administration and uniformity of dosage. The term “dosage unit form” as used herein refers to physically discrete units suited as unitary dosages for the subjects to be treated, with each unit containing a predetermined quantity of heparosan-modified nanoparticles calculated to produce the desired therapeutic effect. The specification for the dosage unit forms of the presently disclosed and/or claimed inventive concept(s) are dictated by and directly dependent on (a) the unique characteristics of the GAG-modified nanoparticles and the particular immune response to be achieved, and (b) the limitations inherent in the art of compounding such an immunogenic molecule.

Aqueous compositions of the presently disclosed and/or claimed inventive concept(s) comprise an effective amount of the nanoparticle, nanofibril, or nanoshell or chemical composition of the presently disclosed and/or claimed inventive concept(s) dissolved and/or dispersed in a pharmaceutically acceptable carrier and/or aqueous medium. The biological material may be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, subcutaneous, intralesional, and/or intraperitoneal routes.

The preparation of an aqueous composition that contains an effective amount of the nanoshell composition as an active component and/or ingredient will be known to those of ordinary skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified. Also, the GAG polymer can be used to enhance a secondary vehicle (e.g., liposomes, nanoparticles, etc.) that acts as a carrier or adjuvant for an immunogenic molecule.

### Definitions

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, and/or CAB. Continuing with this example, expressly included are combinations that contain repeats of one or more item or term, such as BB, AAA, AAB, BBC, AAABCCCC, CBBAAA, CABABB, and so forth. One of ordinary skill in the art will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “heparosan” as used herein will be understood to refer to a carbohydrate chain with a repeat structure of ([-4-N-acetylglucosamine-alpha-1,4-glucuronic acid-beta-1-]ₙ), wherein n is 1 or greater. In certain non-limiting examples, n may be from about 2 to about 5,000. The term “oligosaccharide” generally denotes n being from about 1 to about 11, while the term “polysaccharide” denotes n being equal to or greater than 12. The term “heparosan” may be utilized interchangeably with the terms “N-acetylheparosan” and “unsulfated, unepimerized heparin” and another term “K5 polysaccharide.”

The term “GAG” or “GAG polymer” as used herein refers to a polymer of the glycosaminoglycan (GAG) family. Examples of GAG polymers include heparosan, heparin/heparan sulfate, chondroitin sulfate/dermatan sulfate, keratan sulfate, and hyaluronic acid.

The term “UDP-sugar” as used herein refers to a carbohydrate modified with uridine diphosphate (e.g., UDP-N-acetylglucosamine, UDP-glucuronic acid).

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of nucleotides. Nucleotides, as used herein, will be understood to include deoxyribose nucleotides and/or ribose nucleotides, as well as artificial variants thereof.

The term “analog” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all; in the latter case, these drugs can often act as bait or blockers of activity. Alternatively and/or in addition thereto, for a chemical, an analog may be any structure that has the functionalities (including alterations or substitutions in the core moiety) desired, even if comprised of different atoms or isomeric arrangements.

The term “cargo” as used herein refers to the biologically active component in the HEP-particle composition, while the term “vehicle” as used herein refers to the carrier of the cargo (e.g., the heparosan polymer-coated structure) in the composition.

As used herein, the term “active agent(s),” “active ingredient(s),” “pharmaceutical ingredient(s),” “therapeutic,” “medicant,” “medicine,” “biologically active compound,” “adjuvant” and “bioactive agent(s)” are defined as drugs and/or pharmaceutically active ingredients.

The term “Dalton” (Da) as used herein will be understood to refer to a unit of molecular mass for polypeptides and polysaccharides. The term “kiloDalton” (kDa) as used herein refers to one thousand Daltons. The term “megaDalton” (MDa) as used herein will be understood to refer to one million Daltons (i.e., one thousand kDa).

The term “polydispersity” as used herein refers to a measure of the width of molecular weight distributions of a product. In one, non-limiting example, polydispersity is calculated by dividing the Weight average molar mass (M_{w}) by the Number average molar mass (Mₙ); thus, polydispersity=M_{w}/Mₙ.

The terms “quasi-monodisperse” and “substantially monodisperse” are used herein interchangeably and will be understood to refer to very narrow size distributions approaching the ideal polydispersity value of 1.

The term “PEGylation” as used herein refers to the modification of a molecule by addition of polyethylene glycol thereto.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule/composition can be biologically active through its own functionalities, or may be biologically active based on its ability to activate, modulate, or inhibit molecules/compositions having their own biological activity. In addition, biological activity observed in in vitro proxy models is indicative of in vivo action of a molecule/composition.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including human, domestic and farm animals, nonhuman primates, and any other animal that has mammary tissue.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, or management of a disease and/or condition. The specific amount that is therapeutically effective can be readily determined by the ordinary medical practitioner, and can vary depending on factors known in the art, such as the type of disease/cancer, the patient's history and age, the stage of disease/cancer, and the co-administration of other agents.

A “disorder” is any condition that would benefit from treatment with the compositions disclosed herein. This includes chronic and acute disorders or diseases including those pathological conditions which predispose the mammal to the disorder in question. This includes prophylactically preventing a future disease that predisposes the mammal to the disorder including infectious diseases or degeneration or aging.

The term “effective amount” refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the inventive concept(s). The therapeutic effect may include, for example but not by way of limitation, inhibiting the growth of microbes and/or opportunistic infections. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The terms “administration” and “administering,” as used herein will be understood to include all routes of administration known in the art, including but not limited to, oral, topical, transdermal, parenteral, subcutaneous, intranasal, mucosal, intramuscular, intraperitoneal, intravitreal, and intravenous routes, including both local and systemic applications. In addition, the compositions of the presently disclosed and/or claimed inventive concept(s) (and/or the methods of administration of same) may be designed to provide delayed, controlled or sustained release using formulation techniques which are well known in the art.

The following abbreviations, which may be utilized herein, will be understood to refer the following terms or phrases: Strong anion exchange chromatography [SAX]; PolyAcrylamide Gel Electrophoresis [PAGE]; Curie [Ci]; Deoxyribonucleic acid [DNA]; Ribonucleic acid [RNA]; Poly(ethylene Glycol) [PEG]; Uridine diphosphate [UDP]; Heparan Sulfate [HS]; Dalton [Da]; Kilodalton [kDa]; Molecular Weight [MW]; Molar [M]; Gram [g]; Kilogram [kg]; Milligram [mg]; Microgram [μg or ug]; Nanogram [ng]; Nanometer [nm]; Volt [V]; High Performance Liquid Chromatography-Size Exclusion Chromatography [HPLC-SEC].

### EXAMPLES

Examples are provided hereinbelow. However, the presently disclosed and/or claimed inventive concept(s) is to be understood to not be limited in its application to the specific experimentation, results and laboratory procedures. Rather, the Examples are simply provided as one of various embodiments and is meant to be exemplary, not exhaustive.

### Example 1: Heparosan (HEP) Conjugation

Preparation of heparosan has been described previously, for example in U.S. Published Patent Application No. 2010/0036001, incorporated herein by reference in its entirety. The synthesis of various sized (ranging from about 600 Da to about 4,500 kDa) heparosan polymers are possible using heparosan synthases. These HEP polymers may be modified to carry a functional group (e.g., aldehyde, maleimide, amine, iodoacetyl, pyridyl dithiol/disulfide, sulfhydryl, etc.) which facilitates the heparosan coating process onto various types of nanoparticles to delivering immunogenic molecules to cells.

TABLE 1
EXAMPLES OF HEPAROSAN FUNCTIONAL GROUPS

| Reactive Group “-R” | Reactive Group | Reactive Towards |
|---|---|---|
| —SH | Sulfhydryl | Thiol, maleimide, |
| | | iodoacetyl, |
| | | vinyl sulfone |
| 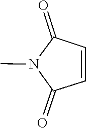 | Maleimide | Sulfhydryl; amino (at high pH) |
| 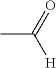 | Aldehyde | Amine, hydrazide |
| 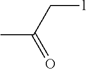 | lodoacetyl | Sulfhydryl |
| 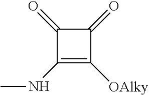 | Mono-squaramide “squarate” | Amine |

Thus, in accordance with the presently disclosed and/or claimed inventive concept(s), there has been provided compositions containing heparosan-particles, wherein the particle serves as the vehicle for carrying a immunogenic molecule, as well as methods of production and use thereof.

As one example of a demonstration, gold (Au) nanoparticles are used as a model system for other particles, monitoring the fate and efficiency of uptake in cell culture models and mice. This invention, however, does not rely on one type of chemistry or particle as this is a cell delivery platform and is only a non-limiting example.

### Experimental Overview

In one embodiment, we covalently attached an orthopyridyl disulfide (OPSS) group via an amide bond to a modified HEP polysaccharide chain containing an amine at the reducing-end terminus to form OPSS-HEP. We selected 10-kDa OPSS-PEG as a comparative control for the 13-kDa OPSS-HEP to match the molecular weights of both polymers.

We modified colloidally dispersed citrate-coated AuNPs with negatively charged HEP polymers as shown in FIG. 1. In FIG. 1, process (A), we performed two different surface modification strategies: (I) an increase in ionic strength to 0.7 M through the step-wise addition of a saline solution (salt aging method, FIG. 1, process (B)), or (II) a single-step pH reduction to pH 3 by addition of an aqueous hydrochloric acid solution (pH reduction method, FIG. 1, process (C)). These two methods were applied to increase surface coating effectiveness by reducing the electrostatic repulsion between individual HEP polymers.

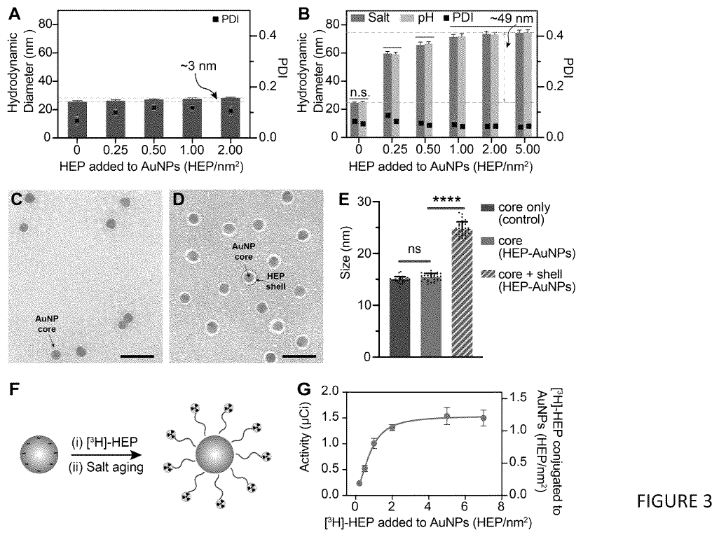

To establish the feasibility of the salt aging and pH methods for the attachment of OPSS-HEP onto AuNPs, 15-nm AuNPs were used as a model nanoparticle system. These AuNPs can be synthesized reproducibly with high yield (typically >80%) and narrow size distribution (<10% deviation). As shown in FIG. 3, Panel A, mixing AuNPs with OPSS-HEP did not result in substantial increases in AuNPs hydrodynamic diameter, measured by dynamic light scattering (DLS), which is likely due to the electrostatic repulsion between individual negatively charged HEP polymers. In contrast, both salt aging and pH methods increased the AuNPs hydrodynamic diameters similarly up to ˜49 nm as a function of the amount of OPSS-HEP added per nanoparticle surface area in a coating reaction (FIG. 3, Panel B). Saturation of the surface was indicated by a plateau when maximal hydrodynamic size was achieved. These DLS results were supported qualitatively by agarose gel electrophoresis experiments, where the migration of the nanoparticles was reduced with an increase in size and HEP surface coverage. Transmission electron microscopy (TEM) of negative-stained nanoparticles indicated the presence of a dense surface coating layer around HEP-modified AuNPs and revealed an average increase in nanoparticle size of ˜25 nm (FIG. 3, Panels C-E). This size increase is smaller than the hydrodynamic size increase observed with DLS, most likely due to a partial collapse of the polysaccharide structure during the sample dehydration process required for TEM imaging.

To determine the HEP coating efficiency on AuNPs, tritium [³H] radiolabeled OPSS-HEP polymers were prepared and used in liquid scintillation counting measurements to quantify the amount of HEP conjugated to AuNPs (FIG. 3, Panels F and G). As shown in FIG. 3, Panel G, the maximum achievable HEP surface coating density was found to be approximately 1.1 HEP/nm². In addition, the colloidal stability of HEP-conjugated AuNPs did not change noticeably for various storage conditions. Collectively, our data confirmed that both salt aging and pH methods resulted in effective and stable HEP surface coating of AuNPs.

Next, we expanded this surface modification strategy to larger nanoparticles to demonstrate the generalizability of modifying particles with GAG polymers. We used both surface modification methods to successfully coat 55-nm and 100-nm AuNPs with OPSS-HEP, resulting in similar overall increases in hydrodynamic diameter of approximately 49 nm was as observed with 15-nm AuNPs. These results indicate that both surface modification strategies were functional and consistent across a wide range of nanoparticle sizes. Additionally, we found that the long-term colloidal stability of AuNPs coated with low HEP surface density (<0.1 HEP/nm²) could be increased to over one year when using the pH method without citrate. In summary, both surface modification strategies allowed the successful coating of HEP polymers onto various particle systems. It is worth mentioning that these surface coating strategies could be used as effective general approaches to modify nanoparticles with negatively charged polymers such as other GAG polymers.

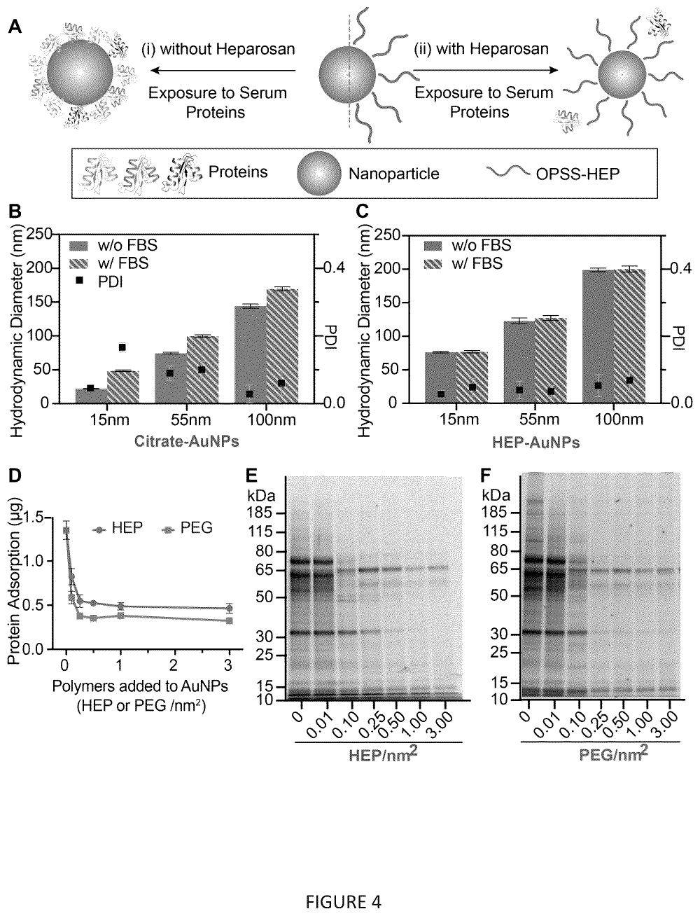

We then analyzed whether HEP would not only enhance the colloidal stability but further reduce serum protein adsorption onto the nanoparticle surface. To evaluate heparosan's ability to reduce protein adsorption, we exposed HEP-modified AuNPs coated at various surface densities to 100% fetal bovine serum (FBS; FIG. 4, Panel A) as a model serum. We qualitatively assessed the serum protein adsorption before and after FBS incubation on 15-, 55-, or 100-nm HEP-AuNPs with two different methods: (i) by DLS via changes in hydrodynamic diameter, and (ii) by agarose gel shift experiments via changes in nanoparticle electrophoretic mobility. After the FBS exposure, nanoparticles without HEP surface modification exhibited a substantial and consistent DLS size increase of approximately 26 nm (FIG. 4, Panel B) and an overall reduced electrophoretic mobility. We did not observe significant differences in hydrodynamic diameter for AuNPs modified with >0.5 HEP/nm² before and after FBS incubation (FIG. 4, Panel C).

To demonstrate the broad applicability of this HEP surface modification strategy, we additionally synthesized HEP-coated silver nanoparticles (AgNPs) and liposomes. We observed no significant changes in hydrodynamic diameter for HEP-coated AgNPs and liposomes before and after FBS incubation indicating minimal interactions between the silver nanoparticle surfaces and serum proteins. Our findings suggest that the HEP surface modification strategy effectively minimizes serum protein adsorption onto the surfaces of various nanoparticles, i.e. AuNPs, AgNPs, and liposomes. We corroborated these findings qualitatively with sodium dodecyl sulphate-polyacrylamide gel electrophoresis (SDS-PAGE) of isolated proteins from AuNPs surfaces (FIG. 4, Panels E-F).

Using a quantitative bicinchoninic acid (BCA) assay, we confirmed that the observed nanoparticle surface protein adsorption correlated inversely with increasing surface density of the HEP coating (FIG. 4, Panel D). This ability to reduce the protein adsorption of HEP-coated AuNPs was similar for AuNPs that were surface-modified with OPSS-PEG. We used PEG as a control surface modification due to PEG's widespread use in nanomedicine. Overall, our findings confirmed that the GAG polymer surface modification using heparosan effectively reduced protein adsorption onto nanoparticles, and this effect was more pronounced with increasing HEP surface coating densities.

Next, we used label-free LC-MS/MS to characterize the adsorbed proteins isolated from the nanoparticle surfaces. Table 1 summarizes the complete list of protein names, molecular weights, and known biological activities of the 16 detected protein species identified on the HEP- and PEG-coated AuNPs. The HEP and PEG surface coatings shared 12 proteins. Average spectral counts for each identified protein from HEP-AuNPs and PEG-AuNPs are reported in Tables 2 and 3, respectively.

TABLE 1
Summary of proteins identified from LC/MS-MS.

| | | MW¹ | |
|---|---|---|---|
| Abbrev | Full Name | (Da) | Biological Process² |
| KNG1 | Kininogen-1 | 71,957 | Blood coagulation, Hemostasis, |
| | | | Inflammatory response |
| HBBF | Hemoglobin fetal subunit | 15,859 | Oxygen transport |
| | beta | | |
| IBP2 | Insulin-like growth factor- | 34,015 | Growth regulation |
| | binding protein 2 | | |
| IGF2 | Insulin-like growth factor II | 19,682 | Carbohydrate metabolism, Glucose |
| | | | metabolism, Osteogenesis |
| HBA | Hemoglobin subunit alpha | 15,184 | Oxygen transport |
| HRG | Histidine-rich glycoprotein | 44,471 | Blood coagulation, Fibrinolysis, |
| | | | Hemostasis |
| CO4 | Complement C4 | 101,885 | Complement pathway, Immunity, |
| | | | Inflammatory response, Innate immunity |
| BPT2 | Spleen trypsin inhibitor I | 10,843 | Protease inhibitor, Serine protease |
| | | | inhibitor² |
| FA5 | Coagulation factor V | 248,983 | Blood coagulation, Hemostasis |
| THRB | Prothrombin | 70,506 | Acute phase, Blood coagulation, |
| | | | Hemostasis |
| APOC3 | Apolipoprotein C-III | 10,692 | Lipid degradation, Lipid metabolism, |
| | | | Lipid transport |
| APOE | Apolipoprotein E | 35,980 | Lipid transport, Transport |
| ALBU | Albumin | 69,293 | Cellular response to starvation, negative |
| | | | regulation of apoptotic process |
| FETUA | Alpha-2-HS-glycoprotein | 38,419 | Acute-phase response, negative regulation |
| | | | of bone mineralization, positive |
| | | | regulation of phagocytosis |
| A1AG | Alpha-1-acid glycoprotein | 23182 | Acute-phase response, regulation of |
| | | | immune system process |
| TSP4 | Thrombospondin-4 | 105974 | Cell adhesion, Tissue remodeling, |
| | | | Unfolded protein response |

¹MW: Molecular weight.
²Molecular function.

TABLE 2
LC-MS/MS analysis of surface adsorbed
proteins from 55-nm HEP-AuNPs.

| | Spectral counts of proteins from HEP-AuNPs at different | | | | | | |
|---|---|---|---|---|---|---|---|
| | coating densities (HEP/nm²) | | | | | | |
| Abbrev | 0 | 0.01 | 0.1 | 0.25 | 0.5 | 1 | 3 |
| CO4 | 0.15 | 0.11 | 0.45 | 0.64 | 0.66 | 0.66 | 0.54 |
| KNG1 | 0.76 | 0.46 | 0.10 | 0.00 | 0.01 | 0.03 | 0.02 |
| HBA | 0.28 | 0.64 | 0.78 | 0.45 | 0.32 | 0.37 | 0.36 |
| THRB | 1.00 | 0.42 | 0.77 | 0.09 | 0.03 | 0.00 | 0.00 |
| IBP2 | 0.32 | 0.79 | 0.67 | 0.41 | 0.03 | 0.01 | 0.06 |
| APOE | 0.33 | 0.98 | 0.21 | 0.00 | 0.17 | 0.05 | 0.00 |
| FETUA | 0.01 | 0.02 | 0.40 | 0.38 | 0.17 | 0.02 | 0.03 |
| HBBF | 0.10 | 0.49 | 0.64 | 0.42 | 0.52 | 0.36 | 0.15 |
| BPT2 | 0.21 | 0.32 | 0.69 | 0.69 | 0.27 | 0.48 | 0.49 |
| IGF2 | 0.14 | 0.50 | 0.62 | 0.00 | 0.00 | 0.00 | 0.00 |
| HRG | 0.43 | 0.43 | 0.67 | 0.00 | 0.00 | 0.00 | 0.00 |
| A1AG | 0.00 | 0.00 | 0.67 | 0.17 | 0.17 | 0.00 | 0.50 |
| ALBU | 0.00 | 0.14 | 0.68 | 0.34 | 0.13 | 0.03 | 0.24 |
| TSP4 | 0.00 | 0.00 | 0.07 | 0.30 | 0.64 | 0.31 | 0.81 |

Total spectral counts are the average of three or four independent replicates.

TABLE 3
LC-MS/MS analysis of surface adsorbed
proteins from 55-nm PEG-AuNPs.

| | Spectral counts of proteins from PEG-AuNPs at different | | | | | | | |
|---|---|---|---|---|---|---|---|---|
| | coating densities (PEG/nm²) | | | | | | | |
| Abbrev | 0 | 0.01 | 0.1 | 0.25 | 0.5 | 1 | 3 | 5 |
| KNG1 | 0.75 | 0.59 | 0.38 | 0.09 | 0.12 | 0.04 | 0.11 | 0.03 |
| HBBF | 0.57 | 0.30 | 0.67 | 0.04 | 0.12 | 0.34 | 0.10 | 0.10 |
| IBP2 | 0.79 | 0.51 | 0.53 | 0.04 | 0.00 | 0.02 | 0.00 | 0.00 |
| IGF2 | 1.00 | 0.52 | 0.48 | 0.00 | 0.00 | 0.00 | 0.00 | 0.00 |
| HBA | 0.44 | 0.33 | 0.90 | 0.24 | 0.07 | 0.41 | 0.22 | 0.45 |
| HRG | 0.83 | 0.33 | 0.00 | 0.00 | 0.00 | 0.00 | 0.00 | 0.00 |
| CO4 | 0.20 | 0.15 | 1.00 | 0.00 | 0.04 | 0.00 | 0.00 | 0.00 |
| BPT2 | 0.06 | 0.06 | 0.55 | 0.26 | 0.30 | 0.38 | 0.41 | 0.08 |
| FA5 | 1.00 | 0.33 | 0.67 | 0.00 | 0.00 | 0.00 | 0.00 | 0.00 |
| THRB | 0.80 | 0.44 | 0.88 | 0.00 | 0.00 | 0.02 | 0.02 | 0.00 |
| APOC3 | 0.08 | 0.17 | 0.50 | 0.50 | 0.50 | 0.67 | 0.42 | 0.42 |
| APOE | 0.53 | 0.19 | 0.77 | 0.07 | 0.06 | 0.33 | 0.39 | 0.09 |
| ALBU | 0.05 | 0.00 | 0.27 | 0.02 | 0.03 | 0.36 | 0.35 | 0.44 |
| FETUA | 0.13 | 0.09 | 0.47 | 0.09 | 0.20 | 0.48 | 0.22 | 0.41 |

Total spectral counts are the average of three or four independent replicates.

The spectral counts varied with both the densities and types of surface coating. We performed hierarchy clustering to organize proteins into groups based on the correlation of relative abundances and observed that the identified proteins presented distinct preferential surface adsorption as a function of the nanoparticle surface coating types and densities. Our proteomic analysis showed that changes in nanoparticle surface coating affect the types and quantities of surface-adsorbed proteins.

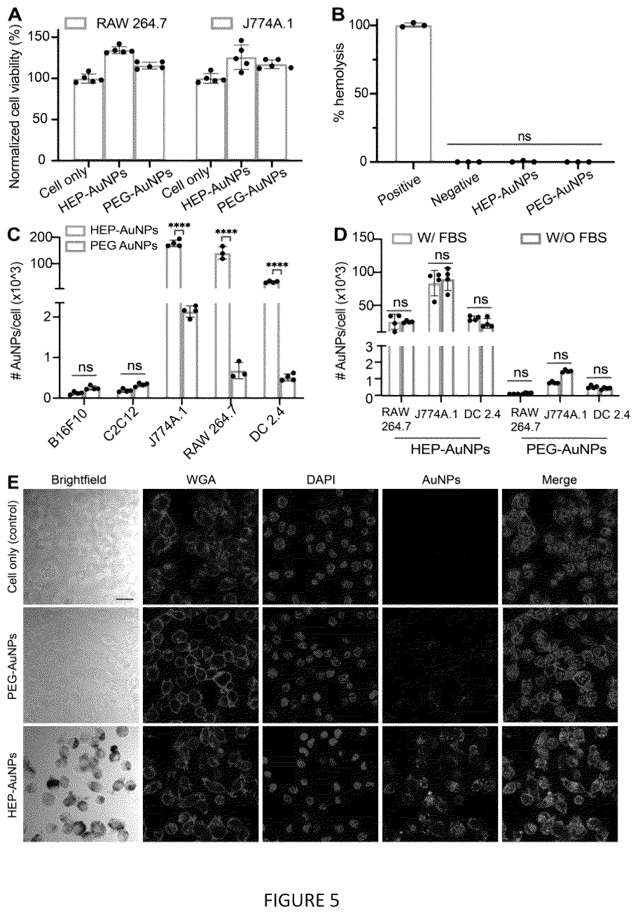

Considering the potential impact of the nanoparticle protein corona on biological function and toxicity, we then compared the cytotoxicity, hemolysis potential, and cytokine release profiles of HEP- or PEG-modified AuNPs (FIG. 5, Panels (A) and (B)). On average, we added about five polymers/nm² in the reactions to modify AuNPs with HEP or PEG. We evaluated the cytotoxicity for various nanoparticle doses, nanoparticle sizes, and incubation periods in different cell types and did not observe any noticeable cytotoxicity at the highest nanoparticle dose tested. In addition, we did not detect any pronounced hemoglobin release upon incubation of human red blood cells with HEP- or PEG-modified nanoparticles (FIG. 5, Panel (B)). We analyzed the cytokine release levels in supernatants of RAW264.7 macrophages after 24 hours of incubation with either citrate-, HEP-, or PEG-modified AuNPs. In comparison to the untreated cell control, no significant changes were observed in this panel of over three dozen cytokines, interleukins, or factors known to be involved in stress and inflammatory reactions. These results highlight the biocompatibility of HEP coatings.

Since the protein corona molecular composition is critical in governing the nanoparticles' biological fate and cellular interactions, we looked at the differences in cell uptake efficiencies between HEP- and PEG-modified nanoparticles. We incubated HEP-AuNPs or PEG-AuNPs with various healthy and cancerous cell lines, including J774A.1 macrophages, RAW264.7 macrophages, DC2.4 dendritic cells, HUVEC human endothelial cells, B16f10 melanoma cells, and C2C12 muscle cells. Our nanoparticle-cell incubation experiments revealed high associations of HEP-AuNPs with certain cell types of the innate immune system. To demonstrate that the observed nanoparticle-cell interactions were due to the intracellular uptake of nanoparticles, we conducted confocal laser scanning microscopy (CLSM) to visualize AuNPs in a label-free manner via light scattering. FIG. 5, Panel (E) shows intracellular CLSM image sections, which confirmed the substantial uptake of HEP-coated AuNPs into these cells.

To further confirm the intracellular localization of AuNPs, we performed a gold etching experiment by exposing cells to KI/I₂ etchant, a highly effective etchant of gold. Our rationale for the etching experiment was that any externally located AuNPs, for example, AuNPs attached to the cell membrane, would dissolve during this etching treatment. We found that quantitative inductively coupled plasma mass spectrometry (ICP-MS) of cells exposed to nanoparticles revealed no notable changes in the extent of nanoparticle cell uptake after the etchant treatment. These results suggest that most AuNPs were located inside the cells in line with our CLSM images. To visualize the subcellular distribution of AuNPs, we performed transmission electron microscopy (TEM) of cells incubated with AuNPs.

We then used ICP-MS to quantify nanoparticle cellular uptake in various cell types. Compared with 55-nm PEG-AuNP, we observed up to 230-fold higher uptake with 55-nm HEP-AuNPs in immune cells, including J774A.1 and RAW264.7 macrophages, as well as in DC2.4 dendritic cells (FIG. 5, Panel (C)). For B16f10 melanoma cells, C2C12 muscle cells, murine 4T1 breast cancer cells, and human endothelial cells (HUVEC), the uptake results of these same HEP-AuNPs were similar to the low levels obtained for PEG-AuNPs (FIG. 5, Panel (C)).

We then looked at the role of the protein corona in driving nanoparticle cell uptake. We performed cellular uptake experiments with and without FBS in the cell media (FIG. 5 Panel (D)). Our results showed that heparosan's intrinsic properties, rather than the protein corona, determined the observed cellular uptake efficiencies.

To investigate whether the nanoparticle size mediated the high cellular uptake of HEP-AuNPs, we incubated RAW264.7 macrophages with 15-nm AuNPs. We discovered a 21-fold higher cell uptake for 15-nm HEP-AuNPs than 15-nm PEG-AuNPs. This finding confirmed that relatively high cell uptake can be achieved even with small HEP-modified nanoparticles and further suggests that HEP has a specific role in driving cellular interactions, and shows improved uptake in comparison to PEG-AuNPs.

Our data suggest that HEP-AuNPs exhibit no apparent cytotoxicity or hemolysis, and display relatively high cellular uptake by specific innate immune cells. There was no correlation found between this high cellular uptake and the protein corona (as shown by controlled FBS incubation experiments), thus suggesting that the uptake behavior is intrinsic to this GAG polysaccharide.

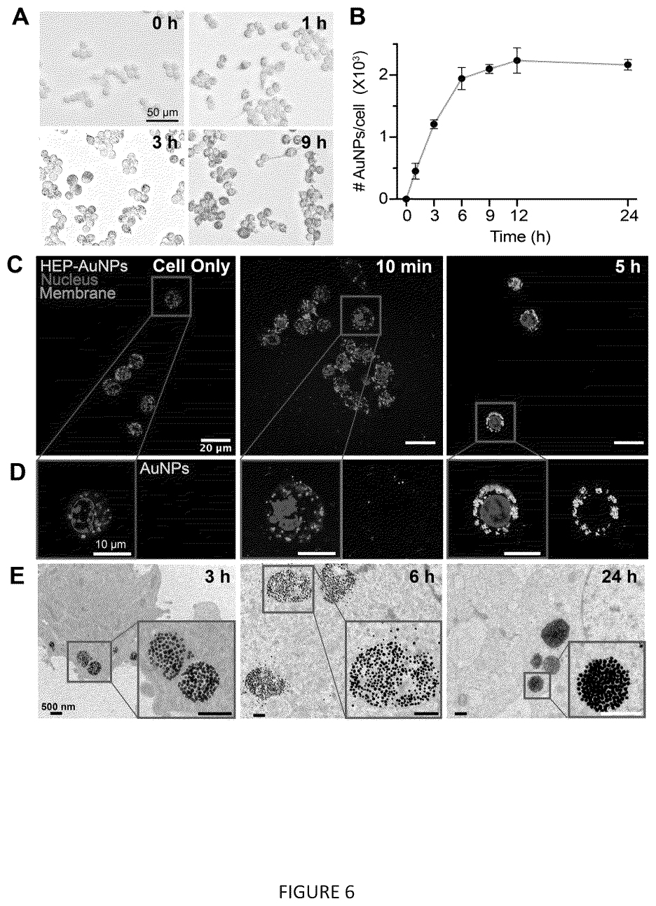

Above, we demonstrated that HEP-coated gold nanoparticles (HEP-AuNPs) efficiently targeted antigen-presenting cells, such as macrophages and dendritic cells, consistent with our previous findings. This study used RAW 264.7 macrophages and DC 2.4 dendritic cells as model immune cells. HEP-AuNPs exhibited a time-dependent nanoparticle uptake behavior when incubated with RAW 264.7 macrophages or DC 2.4 dendritic cells (FIG. 6, Panel (A)). The progressively darker cell coloration (due to the reddish AuNPs) upon brightfield imaging over time suggests an increase in nanoparticle uptake. We quantified the nanoparticle cellular uptake in RAW 264.7 and DC 2.4 cells by inductively coupled plasma mass spectrometry (ICP-MS). We observed that the nanoparticle uptake per cell increased over time, plateauing at ˜12 h post-incubation (FIG. 6, Panel (B)). These results show that innate immune cells exhibited a time-dependent cellular uptake process to internalize HEP-coated nanoparticles.

To further validate the time-dependent cellular internalization, we performed confocal laser scanning microscopy (CLSM) to monitor the nanoparticle uptake behavior in real-time in RAW 264.7 macrophages up to 7 h post-incubation (FIG. 6, Panels (C) and (D)). The HEP-AuNPs were imaged label-free via nanoparticle light scattering and were mainly present surrounding the cell membrane after 1 hour of incubation. We observed strong intracellular nanoparticle signals at 4.5 h, 5 h, and 7 h time points post-incubation. To corroborate the intracellular localization, we subsequently visualized the spatial distribution of nanoparticles in RAW 264.7 macrophages at 3 h, 6 h, and 24 h (FIG. 6, Panel (E)) and DC 2.4 dendritic cells at 3 h and 24 h post-incubation by transmission electron microscopy (TEM). We observed that the HEP-AuNPs were present in intracellular vesicles and discovered that some nanoparticles could escape from these intracellular vesicles to access the cytoplasm (FIG. 6, Panel (E)). Our findings reveal that the cellular uptake of HEP-AuNPs in RAW 264.7 macrophages and DC 2.4 dendritic cells was time-dependent, with the majority of internalized nanoparticles present in intracellular vesicles and a smaller fraction of nanoparticles accessing the cytoplasm.

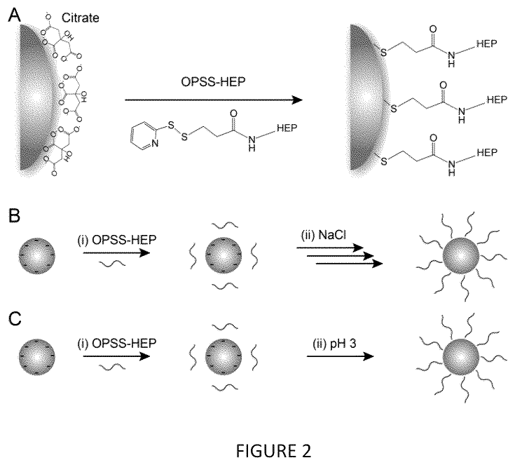

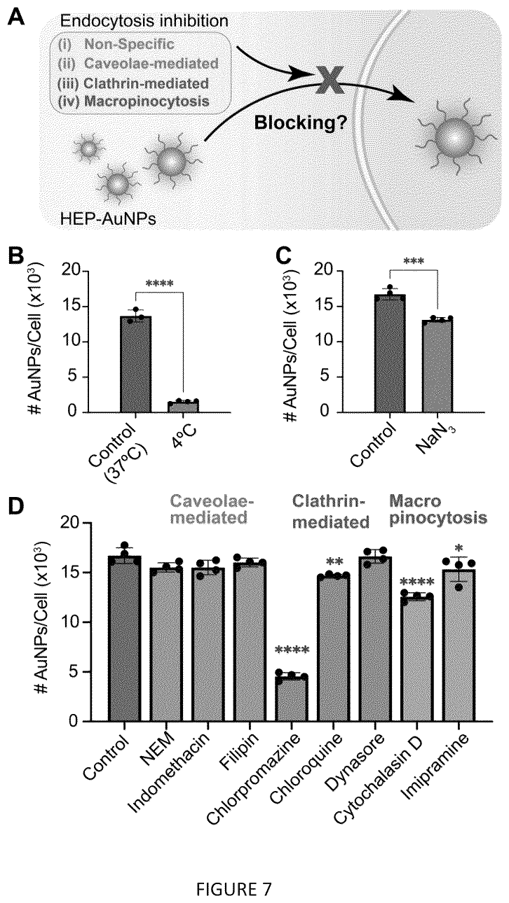

We hypothesized that these nanoparticles might enter cells via endocytosis by one or more energy-dependent uptake pathways. Thus, we carried out a systematic endocytosis inhibition study to discern which uptake pathways were involved. First, we confirmed that energy-dependent endocytosis facilitated the observed nanoparticle uptake by exposing the RAW 264.7 macrophages to known non-specific endocytosis inhibition conditions, i.e. low temperature (4° C.) or 0.1% w/v sodium azide 27-29. We found that the cellular uptake of HEP-AuNPs was reduced by approximately 89% and approximately 22% when the cells were incubated with nanoparticles at 4° C. (FIG. 7, Panel (B)) or treated with sodium azide, respectively (FIG. 2, Panel (C)), confirming an energy-dependent nanoparticle uptake process.

Next, we screened specific endocytosis pathways using established chemical inhibitors that more selectively block endocytosis using known inhibitor concentrations (FIG. 2, Panel (A)). First, we pre-incubated the innate immune cells for 1 h with the endocytosis inhibitors. Then we added the nanoparticles and incubated them with the cells for 1.5 hours. We imaged the cells with a light microscope and quantified the nanoparticle uptake by ICP-MS (FIG. 2, Panel (D)). The ICP-MS results revealed that nanoparticle cellular uptake inhibition efficiencies were approximately 73%, 12%, 24%, or 8% for chlorpromazine, chloroquine, cytochalasin D, or imipramine, respectively (FIG. 2, Panel (D)).

Under our study conditions, the chlorpromazine inhibitor was the most effective agent. As shown in FIG. 2, Panel (D), the endocytosis inhibitors N-ethylmaleimide (NEM), Filipin, Dynasore, and 5-(N-ethyl-N-isopropyl) amiloride (EIPA) did not reduce the nanoparticle cellular uptake. It is known that the cellular uptake machinery and cellular metabolic processes are inter-connected and thus, uptake and transport mechanisms in the context of nanoparticles are difficult to completely define. However, our findings suggest that HEP-AuNPs primarily enter the model innate immune cells through clathrin-mediated endocytosis and micropinocytosis and/or phagocytosis pathways.

We found that chlorpromazine inhibited clathrin-mediated endocytosis while cytochalasin D inhibited macropinocytosis and/or phagocytosis. In our screening experiments, these agents were the most effective HEP-AuNP uptake inhibitors. We performed systematic dose escalation studies to assess the dose-response of the inhibitory effect and the cell toxicity of these two agents. Based on the previous dose screening experiments and published cell viability data the dose ranges were 0-31.4 μM and 0-3.9 μM for chlorpromazine and cytochalasin D, respectively. The cell viability assays confirmed that these inhibitor doses were not cytotoxic under the tested conditions. Using ICP-MS analysis, we quantified the inhibitory effects for nanoparticle uptake in RAW264.7 macrophages to be approximately 70% (chlorpromazine) and approximately 51% (cytochalasin D), respectively. Furthermore, the cell light micrographs showed an apparent reduction in light extinction, consistent with a decrease in nanoparticle cellular uptake. The reduced cellular uptake levels upon chlorpromazine (23.5 μM) and Cytochalasin D (3.0 μM) incubation with RAW 264.7 macrophages were confirmed qualitatively by CLSM imaging. Reduced nanoparticle intensity signals were observed in the cell groups treated with the inhibitors compared to those without the inhibitors.

To test whether the HEP-coated nanoparticles could enter cells through clathrin-mediated endocytosis and macropinocytosis and/or phagocytosis in another immune cell line, we conducted further inhibition experiments in DC 2.4 dendritic cells. Both chlorpromazine and cytochalasin D reduced HEP-AuNP uptake by approximately 77% in DC 2.4 dendritic cells. Additionally, we co-incubated chlorpromazine and cytochalasin D inhibitors with cells to test if there was any additive endocytosis inhibitory effect. However, co-incubation of these two inhibitors showed an approximately 71% inhibitory effect. Thus, significant additive endocytosis inhibition was not observed with this inhibitor combination. We corroborated this finding by co-incubating RAW 264.7 macrophages with both inhibitors. We observed no significant cytotoxicity of the inhibitors at these tested doses. Our results indicate that the cell uptake of HEP-AuNPs occurs in a time-dependent facilitated by clathrin-mediated endocytosis and micropinocytosis and/or phagocytosis.

Our experiments showed that clathrin-mediated endocytosis plays an important role in the cellular uptake of HEP-AuNPs, indicating that specific cell surface receptors may facilitate nanoparticle cell uptake. Since these cell surface receptors are unknown, we wondered whether various structural analogs of HEP polysaccharides, the glycosaminoglycans including heparin, hyaluronan (HA), chondroitin sulfates (CS), could be used as competitors and thereby reduce the uptake of HEP-AuNPs.

To address this question, we pre-incubated RAW 264.7 macrophages systematically with a library of relevant HEP structural analogs as shown below in Table 4 and then added HEP-AuNPs to the cells.

TABLE 4
Summary of HEP Structural Analog Polymers used for Competition Experiments

| | Average | Surface | | | |
|---|---|---|---|---|---|
| | Molecular | Receptors | | Similarity | Difference |
| GAG or | Weight | (not all | Major Repeat | with | from |
| Sugar | (kDa) | inclusive) | Structure | Heparosan | Heparosan |
| Heparosan | 43.8; 169 | unknown | [GlcA]-4-beta- | — | — |
| | | | [GlcNAc]-4-alpha | | |
| Hyaluronic | 160; 1,000 | CD 44; | [GlcA]-3-beta- | same sugar | different |
| acid (HA) | | LYVE-1; | [GlcNAc]-4-beta | composition | glycosidic |
| | | HARE; | | and charge | linkages |
| | | Stabilin-1; | | density | |
| | | Rham | | | |
| Heparan | ~12.9 | Fibroblast | [GlcA]-[6OS- | same | ~1-2 sulfates per |
| Sulfate | | growth factor | GlcNAc/GlcNS] | backbone | repeat |
| | | receptor | | | |
| Heparin | ~16.6 | G6b; Fibroblast | [2S-IdoA/GlcA]- | similar | ~3 sulfates |
| | | growth factor | [6OS-GlcNS] | backbone | per repeat; some |
| | | receptor; FGF2 | | | GlcA epimer, |
| | | | | | IdoA |
| Chondroitin | ~19.5 | CD 44 | [GlcA]-3-beta-[4S- | GAG family | GalNAc instead |
| Sulfate A | | | GalNAc]-4-beta | | of GlcNAc; |
| (CS A) | | | | | different |
| | | | | | glycosidic |
| | | | | | linkages; |
| | | | | | 1 sulfate per |
| | | | | | repeat. |
| Chondroitin | ~21 | | [2S-GlcA/IdoA]-3- | GAG family | GalNAc instead |
| Sulfate B | | | beta-[4,6S- | | of GlcNAc; |
| (CS B) | | | GalNAc]-4-beta | | different |
| | | | | | glycosidic |
| | | | | | linkages; |
| | | | | | ~2 sulfates per |
| | | | | | repeat. |
| Chondroitin | ~45 | CD 44 | [GlcA]-3-beta-[6S- | GAG family | GalNAc instead |
| Sulfate C | | | GalNAc]-4-beta | | of GlcNAc; |
| (CS C) | | | | | different |
| | | | | | glycosidic |
| | | | | | linkages; |
| | | | | | 1 sulfate per |
| | | | | | repeat. |
| Chondroitin | ~39 | | [2S-GlcA]-3-beta- | GAG family | GalNAc instead |
| Sulfate D | | | [6S-GalNAc]-4- | | of GlcNAc; |
| (CS D) | | | beta | | different |
| | | | | | glycosidic |
| | | | | | linkages; |
| | | | | | ~2 sulfates per |
| | | | | | repeat. |
| Chondroitin | ~140 | I. CONTACTI | [GlcA]-3-beta- | GAG family | GalNAc instead |
| Sulfate E | | N-1 | [4,6S-GalNAc]-4- | | of GlcNAc; |
| (CS E) | | | beta | | different |
| | | | | | glycosidic |
| | | | | | linkages; |
| | | | | | ~2 sulfates per |
| | | | | | repeat. |
| Unsulfated | ~100-200 | | [GlcA]-3-beta- | GAG family; | GalNAc instead |
| chondroitin | | | [GalNAc]-4-beta | same charge | of GlcNAc |
| | | | | density | |
| GlcNAc(N- | 0.221 | — | — | monosaccharide | — |
| acetyl- | | | | component | |
| glucosamine) | | | | | |

To quantify the nanoparticles' cellular interactions, we performed quantitative ICP-MS and corroborated the results qualitatively with light microscopy. The ICP-MS and microscopy data both revealed that CS A (i.e. CS with mostly C4-sulfo isomers) was most effective at reducing the cellular uptake (˜43%) of HEP-AuNPs compared with the ‘no-competitor’ group. We observed approximately 15% inhibition by CS C (i.e. CS with mostly C6-sulfo isomers) and approximately 18% inhibition by heparin (i.e. the anticoagulant drug that is a highly sulfated HEP). No significant competition with the remaining structural analogs was observed.

We next investigated whether the CS A inhibitory effect of HEP-AuNP uptake was due to a potential toxicity effect of the CS A preparation, which was extracted from a mammalian source. We observed that the CS A material did not affect cell viability at the working concentrations employed in this study.

Next, we expanded the structural analog competition study to DC 2.4 dendritic cells. Since the previous study demonstrated that CS A significantly reduced uptake of the HEP-AuNPs in RAW 264.7 macrophages, we pre-incubated CS A with the DC 2.4 cells for 1 hour, then added the nanoparticles for an additional 2.5 hour incubation. We quantified the competition efficiency by ICP-MS and corroborated the results with light microscopy. Non-cytotoxic doses of CS A resulted in a lower nanoparticle uptake as quantified by ICP-MS, and we observed a reduced nanoparticle signal compared to the no-competitor group using light microscopy.

We further assessed the competition effect of CS A as a function of time and concentration at non-cytotoxic levels. The CS A agent significantly lowered the cell uptake of HEP-AuNPs, as confirmed by light microscopy and ICP-MS quantification. The inhibitory effect of 1 mg/mL CS A persisted throughout time. At 2 mg/mL, CS A suppressed cellular uptake of HEP-AuNPs up to 9-fold, according to our inhibitor dose-response results (IC50 of 0.5 mg/mL). These competition experiments with GAG structural analog polymers imply that CS A can substitute to some degree as a ligand for HEP for the internalization receptor(s).

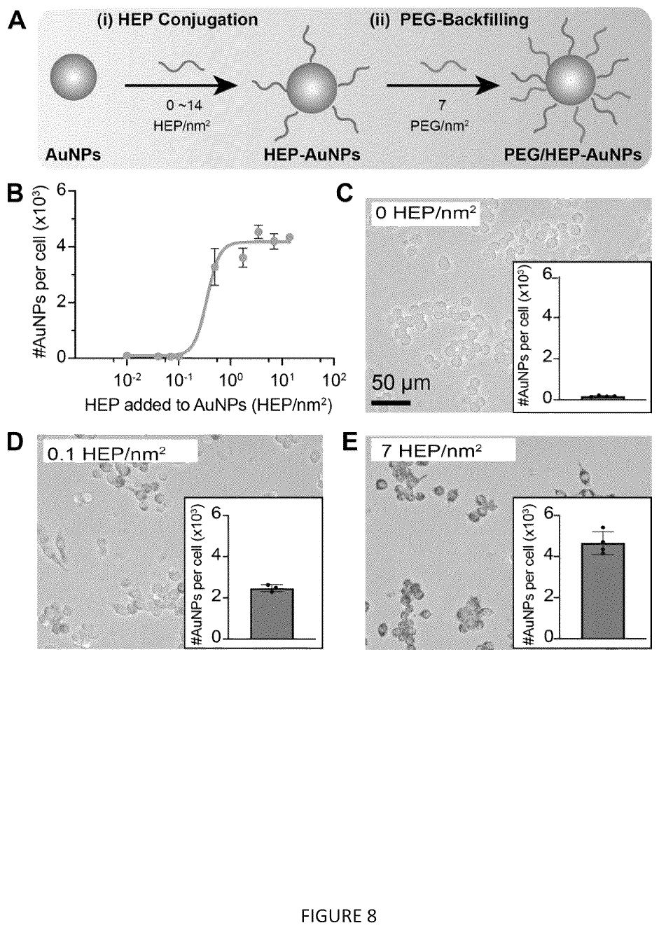

Next, we examined if the substantial cellular uptake of HEP-AuNPs was due to multivalent nanoparticle/receptor interactions by investigating the effect of the HEP surface coating density on internalization. Since uncoated nanoparticles are prone to colloidal instability and substantial protein corona formation that may affect cellular interactions, the nanoparticles were first coated with various amounts of HEP polymers. We then used a backfilling strategy to cover any uncoated surface with methoxy-terminated poly(ethylene glycol), PEG, thereby enhancing nanoparticle colloidal stability (FIG. 8, Panel (A)). PEG is known to minimize non-specific protein adsorption on nanoparticle surfaces, and it is used in the clinic. The coating process was characterized by measuring the hydrodynamic diameter and zeta potential with DLS. The data show that with HEP added at approximately 0.5 HEP/nm², there was no significant difference in the hydrodynamic diameter or the zeta potential values after PEG backfilling. At the added densities of <0.5 HEP/nm², the hydrodynamic diameter and the zeta potential increased with the addition of PEG, indicating that the nanoparticles were successfully backfilled. These results confirm that fully surface-coated nanoparticles with various HEP densities were generated successfully.

Next, we exposed the nanoparticles with various HEP surface coating densities to RAW 264.7 macrophages and evaluated the corresponding uptake efficiencies qualitatively by light microscopy and quantitatively by ICP-MS (FIG. 8, Panels (B) to (E)). We observed that the interaction between the nanoparticles and the cells increased in a HEP surface coating density-dependent manner using light microscopy (FIG. 8, Panels (C) to (E)). We corroborated this observation quantitatively by ICP-MS. Our quantitative results demonstrate that the nanoparticle cellular uptake can be controlled by more than three orders of magnitude via varying the HEP surface coating density (FIG. 8, Panel (B)).

Overall, our results suggest that the multivalent interactions strengthened with increased HEP surface coating density, leading to higher HEP-AuNP cell uptake. Manipulating the surface coating density of GAG polymers may allow for the controlled delivery of nanoparticles to innate immune cells.

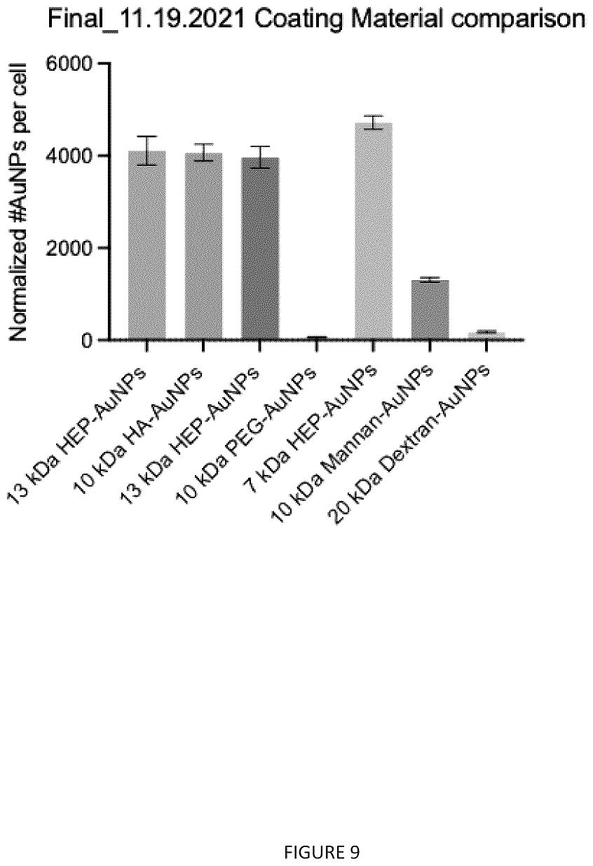

In addition to the above experiments with HEP, as shown in FIG. 9, we have also found that nanoparticles coated with other GAGs, including hyaluronan (HA) and some versions of chondroitin sulfate (CS), can be internalized by immune cells. These GAG-coated nanoparticles were found to work at about the same efficiency as HEP. It is known that the HARE/stabilin receptor can bind HA, CS, and heparin, but not heparosan. The LYVE-1 receptor and CD44 can both bind HA, but not heparosan or CS.

All the GAG-NPs tested were internalized much better than the comparable NPs coated with mannan or dextran or PEG. Mannan and dextran have been proposed to be improved coatings in the literature (Mitchell M J, Billingsley M M, Haley R M, Wechsler M E, Peppas N A, Langer R. Engineering precision nanoparticles for drug delivery. Nat Rev Drug Discov. 2021; 20(2):101-124. doi:10.1038/s41573-020-0090-8). The mannan in particular would be recognized by the mannan-receptor/scavenger receptor that is prevalent in immune cells.

### Example 2: HEP-Nanoparticle Uptake Analysis as Monitored by Cell Culture Experiments

FIG. 5 is a bar chart showing the results of cell culture experiments using gold nanoparticle concentrations of up to 0.3 nM over 6 h. The 13-kDa heparosan polymers were linked to 50-nm gold nanoparticles through an OPSS linker. The 10-kDa PEG polymers were also linked to 50-nm gold nanoparticles through an OPSS linker. The results of FIG. 5 show that highly heparosan-modified nanoparticles exhibit orders of magnitude higher cell uptake in J774A.1 and RAW264.7 macrophages and DC2.4 dendritic cells as compared to PEG-modified nanoparticles. Nanoparticle uptake in HUVEC (human umbilical vein endothelial cells), 4T1 (mammary carcinoma), B16F10 (melanoma), and C2C12 (myoblast) cells was not found to be statistically different between PEG- and heparosan-modified nanoparticles as determined by inductively coupled plasma mass spectrometry (ICP-MS). Light microscopy was then used, as shown in FIG. 6, to confirm the results found for the DC2.4 cells. As shown in FIG. 6, the DC 2.4 dendritic cells took up far more HEP-modified gold particles than PEG-modified gold particles.

Using transmission electron microscopy (TEM), we showed that heparosan-modified nanoparticles were able to escape intracellular vesicular systems to enter the cytoplasm of RAW264.7 macrophages (FIG. 7). This demonstrates that such modified nanoparticles may be useful to deliver immunogenic molecules to the cytoplasm of target cells, such as dendritic cells, macrophages or other white blood cells.

### Nanoparticle Synthesis (15-Nm, 55-Nm, or 100-Nm AuNPs; 55-Nm AgNPs; and Uncoated Liposomes)

A redox reaction-based bottom-up synthesis approach was used for the synthesis of 15-nm, 55-nm, or 100-nm AuNPs. Aqua Regia was used to clean the reaction flasks before synthesis. Aqua Regia is prepared as a 3:1 ratio of hydrochloric acid (Sigma-Aldrich, ACS reagent, 37%) and nitric acid (Sigma-Aldrich, ACS reagent, 70%).

### Synthesis of 15-Nm Gold Nanoparticle

Based on a protocol published by Turkevich et al., we synthesized 15-nm gold nanoparticles (Turkevich, J.; Stevenson, P. C.; Hillier, J. A Study of the Nucleation and Growth Processes in the Synthesis of Colloidal Gold. Discuss. Faraday Soc. 1951, 11 (0), 55-75). Briefly, 98.9 mL nanopure water and 1.0 mL of 0.102M sodium citrate tribasic dihydrate (Sigma-Aldrich) were prepared in aqua regia-cleaned 250 mL Erlenmeyer flask. This flask was then placed on a hot plate with settings of 300° C. and ˜200 rpm. When the mixture solution in this flask started boiling, 100 μl of 0.25 M aqueous gold (III) chloride trihydrate (Sigma-Aldrich) was added rapidly, and the stirring speed was increased to ˜400 rpm. Next, a 7 min timer was set. During this 7 min of reaction, the color of the solution changed from purple to cherry red. After 7 min, the flask was placed on ice to quench the reaction and then stored at 4° C. To prevent nanoparticle aggregation, Tween 20 (Sigma-Aldrich, Molecular Biology, Grade) was added with a final concentration of 0.01% (v/v). To concentrate and wash, nanoparticles were centrifuged at 15,000×g for 90 minutes using a ThermoFisher Heraeus Multifuge X3R centrifuge. Both DLS and UV-Vis spectrophotometry measurements were performed for nanoparticle quality assessment.

### Synthesis of 55-Nm and 100-Nm Gold Nanoparticles

To synthesize larger nanoparticles, a seed-mediated synthesis protocol from Perrault et al. was adopted (Perrault, S. D.; Chan, W. C. W. Synthesis and Surface Modification of Highly Monodispersed, Spherical Gold Nanoparticles of 50-200 Nm. J. Am. Chem. Soc. 2009, 131 (47), 17042-17043). The 15-nm seed gold nanoparticles were prepared by the previously described protocol; these ‘seed’ particles were transferred to a new clean flask to synthesize the two larger AuNPs.

### Synthesis of 55-Nm Silver Nanoparticles

A modified one-pot method was adopted for the synthesis of 55-nm citrate-capped silver nanoparticles (AgNPs). Briefly, tannic acid and sodium citrate tribasic dihydrate were added into 100 mL of boiling nanopure water for final concentrations of 5 mM and allowed to stir vigorously for 15 minutes. Then, 0.1 mL of 250 mM silver (I) nitrate was immediately added to the reaction and boiled for 15 minutes.

### Synthesis of Uncoated Liposomes and PEG-Liposomes

Uncoated liposomes and PEG-coated liposomes were prepared based on a published paper⁴. Briefly, uncoated liposomes with a fluorescent tag for imaging were prepared by adding a stock of 0.44 mg/mL DiO′; DiOC18 (3) (3,3′-Dioctadecyloxacarbocyanine Perchlorate) in chloroform to solid 1,2-distearoyl-sn-glycero-3-phosphocholine (DSPC) and cholesterol (final molar ratio of 1:1.3:0.9, respectively). PEG-liposomes were prepared by using 0.44 mL DiO′; DiOC18(3) (3,3′-Dioctadecyloxacarbocyanine Perchlorate) (solvent is chloroform) dissolved 1,2-distearoyl-sn-glycero-3-phosphocholine (DSPC), cholesterol, and phosphatidylethanolamine modified with 2-kDa polyethylene glycol (DSPE-PEG2000) (final molar ratio of 1:1.3:0.9:0.3). After mixing lipids in the desired ratio, the chloroform was evaporated by a rotary evaporator. The lipid films were suspended in 600 μL of 37° C. warmed 1× phosphate buffered saline using bath sonication (ultrasonic cleaner Branson CPX8800H at 25° C.) for approximately 20 min. The mixture was then extruded through a 100-nm polycarbonate filter at 60° C. for 21 cycles. The hydrodynamic diameter was measured by DLS.

### GAG Reagent Synthesis and Coating

### Heparosan Coating Reagent Synthesis

A quasi-monodisperse 13 kDa-heparosan (HEP) polysaccharide with a polydispersity Mw/Mn 1.038+/−0.005 as measured by HPLC-SEC with multiangle light scattering (Wyatt) detection and a reducing end amino group (HEP-NH2) was synthesized by synchronized, stoichiometrically controlled chemoenzymatic reaction using an amine-containing acceptor, UDP-sugar donors, and PmHS enzyme as described previously.⁵ This starting material was employed to create two derivatives: (a) a HEP with a thiol-reactive group (HEP-OPSS) at the reducing terminus, and (b) a radioactive version of the same polymer tagged at the non-reducing terminus ([³H]HEP-OPSS). HEP polymers were quantified using the carbazole assay with a glucuronic acid standard.

The thiol-reactive dithiol-pyridyl (OPSS) group was introduced into the reducing end of various HEP-NH₂ polymers using a 31- to 42-fold molar excess of N-succinimidyl 3-(2-pyridyldithio)propionate (SPDP) (ThermoFisher) added as 2 or 4 additions in neat DMSO; the reaction was performed with 6-6.7 mg/mL HEP-NH₂ and 30-37% DMSO solvent final in 0.1 M HEPES, pH 7.2, 5 mM EDTA, at room temperature overnight. The HEP-OPSS target was precipitated by the addition of NaCl (0.1 M final) and 4.8 volumes of isopropanol on ice for 2 hours. The resulting pellet was harvested by centrifugation (1,800×g, 30 min), the supernatant was aspirated, and the pellet was dried (3 min under vacuum or air-dried for 2.25 hours) before re-suspension in water at 4° C. overnight. The HEP-OPSS was purified from small MW compounds via either strong anion exchange chromatography or by ultrafiltration.

The HEP-OPSS (˜100 mg synthesis scale) was applied to a HiTrap Q strong anion exchange column (5 mL bed; GE Healthcare) equilibrated in Buffer A (10 mM NaOAc, pH 5.8) at 2 mL/min and washed with 4 column volumes (cv) of 100% buffer A. A series of linear gradient steps with NaCl elution (using B buffer=A+1 M NaCl in steps of 10 cv of 90A:10B, 4 cv of 60A:40B, and then 1 cv of 40A:60B) removed traces of OPSS from the target. The 0.21-0.5 M NaCl SAX fractions containing the HEP-OPSS target were pooled, precipitated with 2.5 volumes of ethanol (similar process to isopropanol employed above), the pellet suspended in water, and stored at −20° C. Alternatively, the HEP-OPSS (˜200 mg synthesis scale) target was purified by repeated ultrafiltration (6 cycles with 3 kDa MWCO membrane; Amicon) against water at room temperature to desalt the sample and to remove any residual SPDP. The presence of the OPSS group on the sugar chain was verified by reaction with SAMSA (a fluorescent thiol activated with base per the manufacturer's instructions; ThermoFisher) and then PAGE analysis.7 A fluorescent band at the appropriate MW was detected, thus indicating the successful installation of the OPSS moiety onto the sugar chain as described later.

Radioactive forms of the HEP-OPSS were created by first end-labeling 100-200 μg HEP-NH₂ with 1.1-9 Ci of UDP-[³H]-GlcNAc (PerkinElmer) and PmHS under reactions conditions similar to nonradioactive HEP-NH₂ synthesis; under these conditions only ˜1-2% of the HEP chains (˜65 monosaccharide units long) are tagged with a single radioactive sugar thus not significantly altering the overall MW of the preparation. The purified material was then reacted with OPSS as above except: (i) a 2,000 to 3,555 molar excess of OPSS was used for 3-4 hrs, (ii) the final concentration of HEP-NH₂ was 0.2 mg/mL, and (iii) the target was precipitated by the addition of NaCl (0.3 M final) and 3 volumes of ethanol at −20° C. for 2 hours. The resulting pellet was harvested by centrifugation (18,000×g for 0.5-1 hr), the supernatant was aspirated, and the pellet was then washed in 70% ethanol/0.1 M NaCl and centrifuged again. The pellet was air-dried, resuspended in water, and then purified by repeated ultrafiltration (6 cycles with 3 kDa MWCO; Amicon) against water. The specific activity of the final [³H]HEP-OPSS product was measured by liquid scintillation counting and determined to be 93-360 mCi/mmol (7-27 nCi/μg).

### Hyaluronan (HA), Mannan, & Dextran Derivatives for NP Coatings

A quasi-monodisperse 13 kDa-Hyaluronan (HA) polysaccharide with a reducing end amino group was synthesized by synchronized, stoichiometrically controlled chemoenzymatic reaction using an amine-containing HA-based acceptor, UDP-sugar donors, and PmHAS enzyme (Jing W, DeAngelis P L Synchronized chemoenzymatic synthesis of monodisperse hyaluronan polymers. J Biol Chem. 2004 279(40):42345-42349). Mannan was obtained from Sigma (base-extracted preparation from the yeast Saccharomyces cerevisiae); it is a glycopeptide so there is a naturally occurring amine attached to the sugar.

The HA and mannan OPSS reagents were made by derivatization with SPDP as for the heparosan-NH₂ and used to coat of gold nanoparticles as described.

Dextran monothiol (10 kDa average MW) as obtained from Fina Biosolutions (Rockville, MD) and used directly for coating the NPs.

### Chondroitin Sulfate Derivatives

Chondroitin sulfate from bovine source (CSA; BioIberica, Barcelona, Spain) or shark source (CSA; BioIberica) was derivatized on a small portion of its carboxylate groups (average ˜1-4/chain target) with a heterobifunctional reagent, PDPH (3-(2-pyridyldithio)propionyl hydrazide)

The reagent PDPH (Thermo) has a hydrazide on one end (reacts with GAG carboxy group) and a OPSS on the other end (reacts with Au or a thiol group). The PDPH (˜1-4 molar equivalents to the CS) in a water stock (˜7 mg/ml) was added to a 10 mg/ml CS preparation in 500 mM BisTris, pH 4.75, at room temperature with mixing. After ˜10 min, a carbodiimide activating reagent, 1-ethyl-3-(3-dimethylaminopropy)carbodiimide hydrochloride (EDC; Sigma) was added as a solid to the solution at ˜2-3 molar excess over PDPH with mixing. After overnight reaction at room temperature, the material was then processed with a Sephadex G-10 column (PD-10; Pharmacia) in 0.1 M NaCl followed by ultrafiltration in water using a spin unit (3K MWCO) to remove the excess reagents. The material was then used to coat the NPs with chondroitin sulfate.

### Polydisperse HA Coating Derivatives

A polydisperse HA preparation (average MW 17 kDa) from a microbial fermentation (Lifecore Biomedical, LLC; Chaska, Minnesota) was treated as for CS and then used to coat the NPs.

### Quantification of HEP- and PEG-Coatings Using DLS for AuNPs and Liposomes

### Saturation Curve of Gold Nanoparticles

Briefly, a constant surface area to volume ratio was maintained for every desired PEG (MW 10 kDa, Laysan Bio) surface density (PEG/nm²); only the surface modification density conditions were varied. The addition ratios of PEG polymer to nanoparticle surface area were 0, 0.1, 0.25, 0.5, 1, 2 PEG/nm² for 15-nm AuNPs. Samples were prepared in triplicate by mixing the DI water, PEG solution, and 15-nm citrate-stabilized AuNPs in order. The vials were vortexed a second time and then left to incubate at room temperature for 30 minutes. After the incubation period passed, the PEG was then fully conjugated to the surface of the nanoparticles, which was verified with the Malvern ZetaSizer using dynamic light scattering (DLS). The DLS measured hydrodynamic diameter, which consists of the gold core diameter and the layer of hydration from the surface-bound molecules. Additionally, the success of the effect of the PEG density on the surface charge of the nanoparticles was qualitatively observed through gel electrophoresis, as described below in the gel electrophoresis section. The heparosan saturation curve was obtained by a similar procedure with the use of the salt aging or pH methods as described below.

### Saturation Curve of Heparosan Coated Liposomes

Naked liposomes were coated with lipid-modified heparosan polymers using post-insertional modification. Briefly, 13-kDa heparosan-dipalmitate polymers were mixed with uncoated liposomes, then incubated for 90 min at 37° C.; these conditions result in efficient incorporation of a HEP-coating on the outer leaflet of the bilayer. The saturation curve was obtained by mixing 9.71 mg/mL heparosan polymer with uncoated liposome at the percentage of molar ratio of HEP polymer to lipids.

### HEP-AuNPs Prepared by the Salt Aging Method

The coating of heparosan by salt aging on 15-nm gold nanoparticles was based on the Hurst/Zhang method. This method entails increasing the concentration of sodium chloride (Sigma) to help the heparosan conjugate attach to the gold nanoparticle surface. Briefly, citrate stabilized AuNPs were obtained that had been prepared as described above. A constant surface area to volume ratio was maintained for every desired heparosan surface density (HEP/nm²); only the surface modification density conditions were varied. The addition ratios of HEP polymer to nanoparticle surface area were 0, 0.25, 0.5, 1, or 2 HEP/nm² for 15-nm AuNPs. Different HEP coating density conditions were performed for 55-nm and 100-nm gold nanoparticles: the range was 0, 0.01, 0.1, 0.5, 1, or 2 HEP/nm². Triplicates were performed for each condition. Nanoparticle and heparosan solution were mixed together in DI water and incubated at room temperature for 20 min. NaCl was added in 0.1 M NaCl increments until the final NaCl concentration reached 0.7 M. Each increment was followed by a 20 min incubation at room temperature before the next addition of NaCl. DLS was performed after the final incubation. Agarose gel electrophoresis was performed as described below in the gel electrophoresis section.

### HEP-AuNPs and HEP-AgNPs Prepared by the pH Method

In a different process from the salt aging method described above, pH 3.0 Citrate-HCl buffer or pH 3.0 HCl without citrate was used as a solvent for the heparosan and gold nanoparticle mixture instead of using DI water. A constant surface area was maintained for every target heparosan surface density (HEP/nm²); only the surface modification density conditions were varied. The addition of HEP polymer to nanoparticle surface reactions were 0, 0.25, 0.5, 1, 2 HEP/nm² for 15-nm AuNPs. HEP surface coating density of 55-nm AuNPs were 0, 0.1, 0.25, 0.5, 1, or 2 HEP/nm². The calculated HEP was added and mixed with acid water, then followed by adding nanoparticles. After a brief vortex, NaCl solution was added in 0.3-M NaCl increments until the final NaCl concentration reached 0.6-M. Each increment was followed by a 20 min incubation at room temperature. DLS was measured after final incubation. Agarose gel electrophoresis was performed as described below in the gel electrophoresis section. The optimized pH method shared the same procedure without the addition of citrate to the acid water. The colloidal stability of the low coating density of HEP was maintained over 390 days with the pH method without citrate addition.

The pH method without citrate addition was used for coating HEP on AgNPs. To attach HEP-OPSS or PEG-OPSS to silver nanoparticles, these reagents were first reduced to HEP-SH or PEG-SH by incubation with Tris(2-carboxyethyl)phosphine hydrochloride (TCEP; Sigma-Aldrich) at a molar ratio of 1:50 for 2 h. This reduction step was employed as the OPSS group does not react efficiently with AgNPs in comparison to AuNPs. The hydrodynamic diameter changes were measured by DLS. Based on the maximum saturation curve we obtained from 55-nm AuNPs by DLS, we added over 5 polymers per nm² in the silver nanoparticle coating.

### Quantification of HEP-Coatings 15-Nm and 55-Nm AuNPs Using a Radiolabeling Strategy

Radioactive heparosan and versions of heparosan-OPSS were mixed in a mass ratio of 1 to 4. This heparosan mixture was used to modify 15-nm or 55-nm AuNPs. By using the salt aging method mentioned above, different densities of heparosan mixture as input surface densities (HEP/nm²) were used to modify 15-nm and 55-nm AuNPs. The input surface HEP densities for 15 nm were 0.2, 0.5, 1.0, 2.0, or 3.0 HEP/nm². For 55-nm AuNPs, the input surface coating reactions were 0.1, 0.25, 0.5, 1.0, or 2.0 HEP/nm 2 After the conjugation process, heparosan-modified AuNPs were centrifuged at 4° C. for 30 min and centrifuged at either 15,000×g for 15-nm or 2,000 xg for 55-nm. To remove free heparosan, the pellet volume after centrifugation was carefully loaded on 25% Percoll (Amersham) and followed by centrifugation at 4° C. (1 h at 15,000×g for 15-nm or 2,000×g for 55-nm AuNPs). The radioactivity was measured by liquid scintillation counting on a Packard Tricarb 2300TR.

### Transmission Electron Microscopy

### TEM Characterization of HEP- and Citrate-AuNPs

Samples were loaded and prepared with negative staining by 2% uranyl acetate (Ted Pella, Inc) on a TEM grid (Ted Pella, Inc). TEM images were taken by a 200-kV field emission JEOL2010F analytical transmission electron microscope with a DE-12 camera. ImageJ (NIH) was used to analyze TEM images.

### TEM Characterization of AuNPs Inside of Cells

RAW 264.7 macrophage cells (˜1 million) were seeded in each well of a 6-well-plate overnight. Dispersions of 0.3-nM PEG- or HEP-AuNPs were then incubated with the cells for 6 h. Any uninternalized AuNPs were removed by washing the cells thrice with 1×PBS. Cells were scraped and collected by centrifugation (500×g, 5 min, 25° C.) into a 1.5-mL microcentrifuge tube. The supernatant was removed, and the cell pellets were fixed with a freshly made fixative solution containing (2% glutaraldehyde: 4% paraformaldehyde (v/v) in 0.2 M cacodylate buffer) at room temperature for 1 hour. Samples were stored at 4° C. until sectioning and negative staining (3% lead citrate solution, cat. 22410, Electron Microscopy Sciences). The TEM micrographs were taken with a Hitachi H-7600 Transmission Electron Microscope.

### Agarose Gel Electrophoresis

Gels with 0.5% (m/v) agarose (Fisher BioReagents) and 0.5×TBE buffer (Sigma-Aldrich) were used to analyze HEP coating on AuNPs. Nanoparticle samples were concentrated to ensure visibility and (typically 10 μL/lane) then mixed with 150 mg/mL Ficoll (Research Products International) in a 4:1 ratio for loading into wells. Gels were run at 50 V for 40 min. Gel images were taken with an Azure C600 imager using visible light.

### Protein Corona Formation, Isolation, and Clean Up

### Protein Corona Formation Upon Nanoparticle Incubation in FBS

Briefly, HEP- or PEG-modified gold nanoparticles were incubated with fetal bovine serum (FBS, ThermoFisher) at a ratio of 10 μL per cm² of nanoparticle surface area. This incubation was at 37° C. for 24 hours, performed in triplicate. To remove unbound FBS, three rounds of washing were performed by 500 μL of 1×PBS with 5-mM EDTA and 0.05% (v/v) Tween 20 at 18,000×g for 30 min at 4° C. After the final wash, the nanoparticles were then measured by DLS and assessed with agarose gel electrophoresis as described in previous sections.

Similarly, we exposed HEP-, PEG-coated AgNPs, or liposomes to FBS, and measured the hydrodynamic diameter change by DLS.

### Protein Isolation

Samples with 50 cm² nanoparticle surface area were prepared for FBS incubation, followed by the incubation and washing protocol described in the previous paragraph. After the final wash, resuspend the nanoparticle pellets in the residual solution (15 L). Next, to isolate proteins from nanoparticles, 8 μL of the 4×LDS buffer (Invitrogen) and 4 μL of the 0.5-M dithiothreitol (DTT) solution were added to the vials. The vials were then incubated at 70° C. for 60 minutes to strip the proteins bound to the surface of the nanoparticles. After the 60-minute incubation, the vials were centrifuged at 18,000×g for 15 minutes to remove nanoparticles. Around 30 μL protein supernatant was collected from each tube; 6.5 μL was reserved for SDS-PAGE. The rest of the proteins were processed with clean-up to remove DTT and LDS.

### Protein Cleanup

To remove the DTT and LDS in the remaining protein solutions, the trichloroacetic acid (TCA)/acetone method was used. Proteins were precipitated by the addition of 950 μL 10% w/v TCA (Sigma) in acetone (ThermoFisher) overnight at −80° C. The next day, the precipitated proteins were collected by centrifugation at 18,000×g for 15 min at 4° C., and the supernatant was discarded. The pellets were first dissolved in 500 μL of 0.03% w/v sodium deoxycholate (Sigma) and then incubated on ice for 30 min after adding 100 μl of 72% (w/v) TCA. The supernatant was removed after centrifugation at 18,000×g, 4° C. for 15 min. The pellets were dissolved in 1 mL of acetone. The 1 mL solution was split into aliquots of 400 μL for BCA assay and 600 μL for LC-MS/MS and dried in a fume hood. The pellets were stored at −80° C. until LC-MS/MS characterization.

### SDS-PAGE for Protein Corona Characterization

SDS-PAGE gels procedures were based on protocols from Walkey et al. (Walkey, C. D.; Olsen, J. B.; Song, F.; Liu, R.; Guo, H.; Olsen, D. W. H.; Cohen, Y.; Emili, A.; Chan, W. C. W. Protein Corona Fingerprinting Predicts the Cellular Interaction of Gold and Silver Nanoparticles. ACS Nano 2014, 8 (3), 2439-2455). We used 4-12% NuPAGE™ Bis-Tris precast Protein Gels, 1.0 mm, 12-well (ThermoFisher) with as a PageRuler™ Plus Prestained 10-250 kDa Protein Ladder (ThermoFisher) standards in a mini gel tank (ThermoFisher) for SDS-PAGE. The 6.5-μL samples previously saved (section 11) were then mixed with 2.5 μL of the 4×LDS buffer and 1 μL of the 500 mM DTT solution and incubated for 5 minutes at 95° C. Along with 2 μL of the protein ladder, samples were then carefully injected into the wells on the gel, and the gel was run at 200 V for 55 minutes on ice. Once the gel was done, it was carefully separated from the case, and the gel was submerged in the fixing solution (10% (v/v) acetic acid (Fisher Scientific) and 40% (v/v) ethanol (PHARMCO-AAPER)) in a petri dish overnight at room temperature with gentle agitation. The next morning, the gel was rinsed with DI water and then stained by 1×SYPRO™ Tangerine Protein Gel Stain according to the manufacturer's protocol for 60 minutes at room temperature with gentle agitation (wrapped in aluminum foil to avoid light). Stained gel was rinsed with DI water and imaged under Azure C600 with an excitation/emission set compatible with the stain and ladder. ImageJ (NIH) was used to analyze the intensity of each lane on the same SDS PAGE images.

### Liquid Chromatography Tandem Mass Spectrometry (LC-MS/MS)

### Liquid Chromatography Tandem Mass Spectrometry (LC-MS/MS)

The protein pellet was solubilized in 15 μL of 25-mM ammonium bicarbonate. Solutions with 6 M urea, 200 mM dithiothreitol, and 200 mM iodoacetamide was prepared in 25 mM ammonium bicarbonate. The protein solution was incubated with 1 μL of 6 M urea and 1 μL of 200 mM dithiothreitol for 1 h at 37° C. for denaturation and reduction. Then the reduced proteins were incubated with 5 μL of 200 mM iodoacetamide for 30 minutes in the dark at room temperature for alkylation. After incubation, 5 μL of 200 mM dithiothreitol was added to the solutions followed by incubation with 3 μL of 0.1 μg/L trypsin (prepared in 25 mM ammonium bicarbonate) at 37° C. and pH 7 overnight. For both the HEP coating and PEG coating, protein digest samples containing varying coating densities were prepared in triplicate. All samples were stored at −20° C. until analysis.

A 15-μL aliquot of the protein digest was injected onto a custom-packed C18 reverse-phase liquid chromatography (RPLC) column (75 m i.d., 150 mm length, 2 m C18 resin) for peptide separation. Mobile phase A was 0.1% formic acid in HPLC grade water and mobile phase B was 0.1% formic acid in acetonitrile. The flow was split to result in a flow rate of approximately 0.8 μL/min through the RPLC column. The LC gradient started with sample loading at 0% mobile phase B for 30 min, followed by an increase from 0% to 35% mobile phase B over 120 min. The mobile phase B gradient was increased to 90% over 3 min and was held constant for 5 min. Mobile phase B was then decreased to 0% over 2 min and maintained for 50 min for column re-equilibration. The eluted peptides were analyzed using an LTQ mass spectrometer (Thermo Fisher Scientific, Hanover Park, IL, USA) with a custom nano-ESI interface. The heated capillary temperature was 275° C. with a spray voltage of 3.5 kV. MS scans were obtained with a normal scan rate and the m/z range was 350-1350. MS/MS scans were acquired using ITMS with collisional induced dissociation (CID) at a normalized collision energy setting of 35%. The ten most abundant precursor ions were selected for MS/MS. The AGC for MS/MS was 3E4 and the maximum ion injection time was 50 ms with 3 microscans. The column was washed between sample runs by injecting a buffer blank and running the same gradient setup. Peptides were identified using MSGF+ to search the mass spectra from the LC-MS/MS analysis against the annotated bovine database downloaded from www.uniprot.org (proteome ID is UP000009136).

### Example 3: Administering GAG-Particle with Bound Immunogenic Peptide as a Vaccine

For a NP vaccine, cargo is typically an antigen or a nucleic acid encoding an antigen. Antigen examples include synthetic peptides, recombinant or native foreign proteins, and microbial polysaccharides. Strategies for antigen incorporation into NPs include loading (i) into the PLGA cores during the emulsification stage or (ii) into the lumen of liposomes with the solution that is used to rehydrate the lipid films. In this strategy, the GAG or heparosan coating may then be added after immunogenic molecule loading of the NPs.

The GAG-coating directs efficient uptake into the immune cell (e.g., dendritic cell, macrophage). The antigenic protein is presented to other cell types of the immune system (e.g., CD8+ and CD4+ T-cells) thus triggers the adaptive immune response needed for the vaccine. This response ultimately results in the production of antibodies (e.g., IgM, IgG) and specific T-cells directed against the antigen in the patient that fight the disease or kill the pathogenic microbe.

In current mRNA vaccines, a ‘packaging’ system (typically a polycation or cationic lipid) is used to bind and stabilize the message encoding an antigenic protein in a liponanoparticle (LNP). Our invention employs a GAG coating on the particle to enhance cellular uptake in a targeted and more efficient fashion.

In one embodiment, the HEP-lipid may be added during the LNP formulation process to form GAG-particles that deliver the vaccine to immune cells. Basically, the typical LNP manufacturing process combines two fluid phases in a rapid fashion: (i) a water-based solution with the mRNA and packaging agent, and (ii) a water-miscible solvent (e.g., ethanol) with the lipids. Depending on the GAG-reagent's solubility (effected by lipid component identity and the sugar chain size), the coating molecule can be dissolved in either phase and the exact choice is not critical for this invention. Upon mixing all the components, the LNPs then self-assemble with the mRNA in the interior and the GAG exposed on the surface.

The resulting mRNA-loaded GAG-NPs are purified by centrifugation, gel filtration, dialysis, or tangential flow filtration to remove the solvent and other reaction components from the LNP formulation. These purification methods are also useful for formulation adjustment (e.g., pH with buffer ion, osmotic pressure with solutes, sterility with preservatives, etc) to make appropriate for administration to the mammalian patient. In one example, intramuscular injection introduces the HEP-NP mRNA vaccine into the environment of immune cells. A GAG-coating directs more efficient uptake into the immune cell, but not the muscle cell. The mRNA escapes into the cytoplasm and is translated into the antigenic protein thus starts the adaptive immune response needed for the vaccine efficacy. Due to the nature of the mammalian immune response, higher titers of anti-antigen antibodies and antigen-specific T-cells (i.e. boosting) are achieved by injecting multiple doses of the GAG-LNP-mRNA vaccine weeks or months apart.

### Bioassay of HEP-NP Peptide Antigen Constructs.

The major histocompatibility complex (MHC) receptors are used to present antigens and train the T cells of the adaptive immune response. Two validated methodologies, flow cytometry and ELISPOT, verify the efficacy of the HEP-NPs loaded with an antigenic peptide (e.g., a 9 amino acid residue long synthetic compound corresponding to the vaccination target disease or microbe) to functionally stimulate the presentation of antigen/MHC complexes on the dendritic cell surface (Hawkins O E, Vangundy R S, Eckerd A M, Bardet W, Buchli R, Weidain, J A, Hildebrand W H. Identification of breast cancer peptide epitopes presented by HLA-A*0201. J Proteome Res. 2008 7 (4): 1445-57).

First, DC 2.4 cells are incubated with HEP-coated, peptide-loaded nanoparticles. After ˜6-72 hrs, flow cytometry with peptide/MHC antibodies is used to assess the amount of NP cargo processed by the immune cells (e.g., dendritic cells) and physically presented by MHC on the surface of the cells. Then ELISPOT assays are used to demonstrate that these peptides activate T cells. Basically, DC 2.4 cells are incubated with HEP-coated, peptide-loaded NPs and IL-2 followed by plating of the cells on anti-IFN-7-coated 96-well plates. Plates are developed and read on a plate reader to determine the extent of T cell activation. The average of the number of spots from the 12 wells of cells treated with irrelevant peptide (negative control) plus two standard deviations is used as the cutoff value. Averages from test wells re-stimulated with individual epitope candidate peptides that are above the cut-off value from the negative controls will be considered as positive. The positive result indicates that immune cell training has occurred with the HEP-NPs, one of the indications of efficacy of a vaccine.

What is claimed is:

1. A composition comprising:
at least one glycosaminoglycan polymer bound to a particle; and
at least one immunogenic molecule covalently or non-covalently linked to the particle.

2. The composition of claim 1, wherein the particle is a nanoparticle.

3. The composition of claim 1, wherein the glycosaminoglycan polymer has a mass in a range of from about 600 Da to about 100 kDa.

4. The composition of claim 1, wherein the glycosaminoglycan polymer is a heparosan polymer.

5. The composition of claim 4, wherein the heparosan polymer is a linear chain heparosan polymer.

6. The composition of claim 1, wherein the at least one immunogenic molecule is a nucleic acid encoding an immunogenic peptide or protein.

7. The composition of claim 6, wherein the nucleic acid is ribonucleic acid.

8. The composition of claim 1, wherein the composition is a vaccine composition.

9. The composition of claim 1, wherein the particle is a gold particle, a silver particle, a polymeric particle or a liposome particle.

10. A method of targeting an immunogenic molecule to an immune cell, comprising:
providing the composition of claim 1,
contacting the composition with an immune cell; and
incubating the composition and immune cell so that the composition crosses the cell membrane and enters the immune cell.

11. The method of claim 10, wherein contacting the composition with the immune cell is performed in vivo.

12. The method of claim 10, wherein contacting the composition with the immune cell is performed ex vivo.

13. The method of claim 10, wherein the composition comprises a heparosan polymer linked to a lipid-containing or polymeric particle, and wherein the lipid-containing or polymeric particle comprises the immunogenic molecule.

14. The method of claim 13, wherein the immunogenic molecule is an antigenic peptide.

15. The method of claim 10, wherein the composition comprises a nucleic acid.

16. The method of claim 15, wherein the nucleic acid is a ribonucleic acid.

17. The method of claim 10, wherein the composition is a vaccine composition.

18. The method of claim 10, wherein composition comprises a particle, and the particle is a gold particle, a silver particle, a polymeric particle or a liposome particle.
